(19) United States
(12) Patent Application Publication
(10) Pub. No.: US 2025/0075025 A1
(43) Pub. Date: Mar. 6, 2025

(54) POLYURETHANE ELASTOMER, MODIFIED SILICONE RUBBER, ACOUSTIC LENS, AND ULTRASONIC PROBE

(71) Applicant: WUHAN UNITED IMAGING HEALTHCARE CO., LTD., Wuhan (CN)

(72) Inventors: Haojie Wei, Wuhan (CN); Kang Si, Wuhan (CN); Cong Liu, Wuhan (CN)

(21) Appl. No.: 18/814,653

(22) Filed: Aug. 26, 2024

(30) Foreign Application Priority Data
Aug. 30, 2023 (CN) 202311123830.9

(51) Int. Cl. C08G 18/66; A61B 8/12; C08G 18/10; C08G 18/32; C08G 18/40; C08G 18/48; C08G 18/61; C08L 83/04; G10K 11/30

(52) U.S. Cl. CPC C08G 18/6674; A61B 8/12; C08G 18/10; C08G 18/3206; C08G 18/4009; C08G 18/4854; C08G 18/61; C08L 83/04; G10K 11/30; C08L 2205/08; C08L 2312/08

(57) ABSTRACT

The present application relates to a polyurethane elastomer, modified silicone rubber, an acoustic lens, and an ultrasonic probe. The polyurethane elastomer includes a structural unit represented by formula I and a chain extender unit, wherein the structural unit is a polyurethane main chain structural unit:

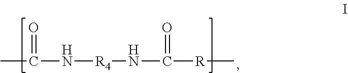

where R is selected from structural units represented by formula I-1 and/or formula I-2 and/or formula I-3

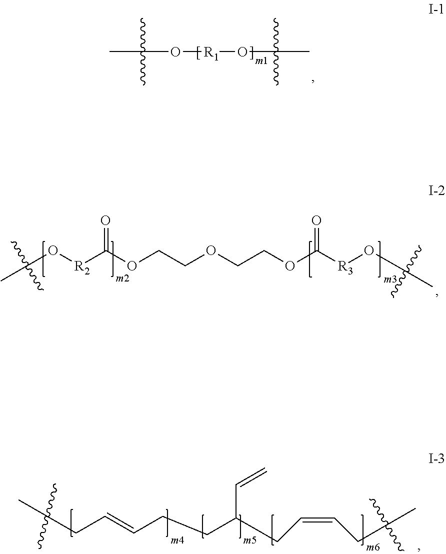

where R₁, R₂, and R₃ are each independently selected from C2-C10 hydrocarbon chains; a number average molecular weight of the structural unit represented by each of the formula I-1, formula I-2, and formula I-3 is ranged from 400 to 5000; and a molar ratio of the chain extender unit to the structural unit represented by formula I is ranged from 0.3:1 to 0.8:1.

### CROSS-REFERENCE TO RELATED APPLICATIONS

This application claims priority to Chinese patent application No. 202311123830.9, filed on Aug. 30, 2023, the content of which is hereby incorporated by reference in its entirety.

### TECHNICAL FIELD

The present application relates to a polyurethane elastomer, modified silicone rubber, an acoustic lens, and an ultrasonic probe.

### BACKGROUND

Medical ultrasound imaging is currently one of the most commonly used clinical imaging diagnostic methods, and its image quality largely depends on the performance of an ultrasound transducer. The ultrasound transducer is a device that converts electrical energy into ultrasonic energy, also known as a sound wave generator or electrode. The ultrasound transducers are typically made of piezoelectric crystal materials, which can convert electrical signals into mechanical vibrations, and then convert mechanical vibrations into ultrasonic signals. The factors affecting the performance of the ultrasound transducers are complex and varied, ranging from the piezoelectric materials themselves to the corresponding acoustic materials, such as the backing material, the matching layers, and the acoustic lenses. As the outermost acoustic component of the ultrasound transducer, the acoustic lens not only protects the internal structure and blocks external contamination and damage, but also controls and adjusts the focusing and diffusion of ultrasound waves, allowing ultrasound waves to focus or concentrate energy in a specific area. This control can achieve higher resolution, reduce interference and noises, and improve imaging quality. Additionally, the acoustic lens can increase the energy density of ultrasound waves, accelerate signal transmission speed, and enhance detection accuracy and sensitivity. If there is a significant difference between the acoustic impedance of the acoustic lens and the acoustic impedance of the detection target, a larger proportion of ultrasound waves will be reflected by the surface of the detection target, preventing the ultrasound waves from effectively entering the target, ultimately resulting in a lower energy utilization rate of the transducer. Therefore, in developing acoustic lens materials, it is needed not only to reduce the acoustic attenuation of the material but also to match the acoustic impedance of the acoustic lens material with the acoustic impedances of the detection target and the matching layer.

Silicone rubber processes characteristics such as softness, high-temperature resistance, corrosion resistance, and excellent biocompatibility. Additionally, silicone rubber has a low sound velocity (about 1000 m/s) and a low acoustic attenuation (0.3 to 0.4 dB/mm/MHz), making it a long-standing material for forming acoustic lenses in ultrasound transducers. However, the low density of silicone rubber (1.0 to 1.3 g/cm³) results in a low acoustic impedance (1.0 to 1.2 MRayl), which differs to some degree from the acoustic impedance of the human body (about 1.5 MRayl). For example, room temperature vulcanizing silicone rubber RTV630 has a density of about 1.27 g/cm³, which is relatively high for silicone rubber, and has an acoustic attenuation at 2 MHz of about 10.04 dB/cm, which is also higher than ordinary silicone rubber. However, the acoustic impedance of RTV630 is only about 1.3 MRayl, still failing to match the acoustic impedance of the human body. Therefore, in order to achieve the desired acoustic impedance, a large amount of inorganic filler needs to be added to the silicone rubber, as indicated in Chinese patent application publication No. CN109922736A. However, this approach causes ultrasonic scattering due to the filler particles, leading to increased acoustic attenuation. In recent years, some patent documents have proposed using resin-modified silicone rubber to prepare acoustic lens materials that meet requirements, such as Chinese patent application publication No. CN111698946A, which suggests using polysiloxane grafted with resins containing ester bonds, amide bonds, urethane bonds, and other functional groups to prepare acoustic lens materials with suitable acoustic impedance. However, this approach involves complex synthesis steps, and the final molding requires crosslinking under radiation, making the process quite challenging.

Polyurethane is a general term for macromolecular compounds containing repeating urethane groups (—NHCOO—) in the main chain, formed from polymerization of a diisocyanate or a polyisocyanate with a dihydroxy or polyhydroxy compound. Polyurethane has a relatively high sound velocity (about 1500 m/s), which is close to the sound velocity in the human body (1540 m/s). Therefore, acoustic lenses using polyurethane has to be designed with a large curvature, which may affect normal use.

Mutual modification between organosilicon and polyurethane is a common approach in blending of high-temperature vulcanizing silicone rubber with thermoplastic polyurethane or in modification of polyurethane with an organosiloxane oligomer.

For example, Chinese patent application publication No. CN105924972A provides silicone rubber/polyurethane thermoplastic vulcanized silicone rubber and a preparation method thereof. However, an ethylene vinyl acetate (EVA) or maleic anhydride grafted modified silicone rubber is used as a compatibilizer, and a filler is added to the silicone rubber to increase viscosity, so as to improve the compatibility and viscosity ratio between the silicone rubber and polyurethane. By adopting dynamic vulcanization technology, the prepared thermoplastic vulcanizate has a fine structure, excellent wear resistance, silky touch, easy adhesion, and excellent mechanical properties and elasticity. However, this material cannot meet the acoustic performance requirements for acoustic lenses.

Another example is Chinese patent application publication No. CN111979791A, which provides a preparation method of silicone-modified hyperbranched polyurethane. Methylvinyl terminated silicone rubber is modified with a polyurethane silane coupling agent, producing polyurethane-based modified silicone rubber, which is used as an anti-aging silicone rubber material for producing three-dimensional printed letters on elastic fabrics. This material not only has weather resistance and other physical properties similar to mixed silicone rubber but also tightly bonds silicone rubber with high-elastic fibers, enhancing the bonding strength at the contact interface, allowing long-term use on high-elastic fabric surfaces without cracking or peeling issues. However, it does not involve optimization of acoustic performance.

Additionally, Canadian patent application publication No. CA2464455A1 provides a thermoplastic elastomer prepared by mixing organosilicon and thermoplastic polyurethane. The corresponding series products DUPONT™ TPSiV have been introduced to the market by Dow Corning Corporation, the applicant of the patent document, for years. However, since these products are simply formed by incorporating vulcanized silicone into a thermoplastic matrix, distinct phase separation is still observed in their micrographic structure. Testing of their commercial samples reveals that their acoustic attenuations at 2 MHz reach 27.8 dB/cm, so these products are not suitable for being used as acoustic lens materials.

### SUMMARY

A polyurethane elastomer includes a structural unit represented by formula I and a chain extender unit, wherein the structural unit represented by formula I is a polyurethane main chain structural unit:

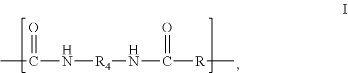

where R₄ is C6-C20 arylene or C6-C20 cycloalkylene, and R is selected from structural units represented by formula I-1 and/or formula I-2 and/or formula I-3:

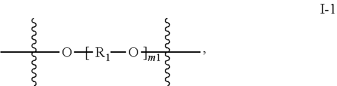

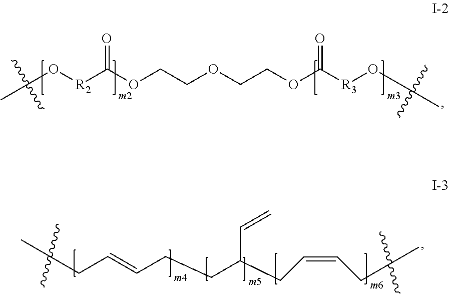

where R₁, R₂, and R₃ are each independently selected from C2-C10 hydrocarbon chains, and m1, m2, m3, m4, m5, and m6 are all positive integers;
a number average molecular weight of the structural unit represented by formula I-1 is ranged from 400 to 5000, a number average molecular weight of the structural unit represented by formula I-2 is ranged from 400 to 5000, a number average molecular weight of the structural unit represented by formula I-3 is ranged from 400 to 5000; and
a molar ratio of the chain extender unit to the structural unit represented by formula I is ranged from 0.3:1 to 0.8:1.

A raw material mixture for preparing a polyurethane elastomer, includes a first prepolymer component and a second prepolymer component;
the first prepolymer component includes a first raw material A, a catalyst, and a chain extender with a chain extension coefficient ranged from 0.3 to 0.8;
the first prepolymer component and the second prepolymer component satisfy an isocyanate index R ranged from 0.8 to 1.2;
where:
the first raw material A includes a raw material of formula IV-1, formula IV-2 or formula IV-3:

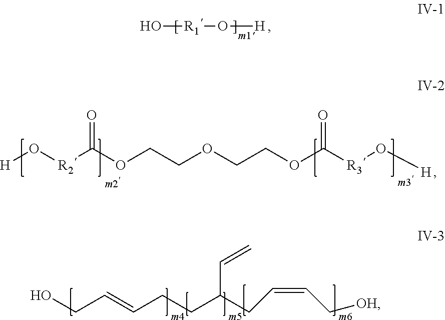

where R₁′, R₂′, and R₃′ are each independently selected from C2-C10 hydrocarbon chains, and m1′, m2′, m3′, m4, m5, and m6 are all positive integers;
a number average molecular weight of the raw material of formula IV-1, formula IV-2, or formula IV-3 is ranged from 400 to 5000;
the structure of the second prepolymer component is represented by formula V:

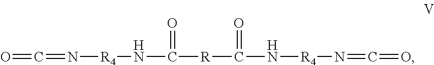

where R₄ is C6-C20 arylene or C6-C20 cycloalkylene, and R is selected from structural units represented by formula I-1 and/or formula I-2 and/or formula I-3:

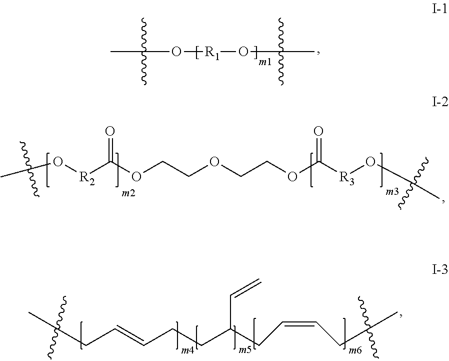

where R₁, R₂, and R₃ are each independently selected from C2-C10 hydrocarbon chains, and m1, m2, m3, m4, m5, and m6 are all positive integers; and
a number average molecular weight of the structural unit represented by formula I-1 is ranged from 400 to 5000, a number average molecular weight of the structural unit represented by formula I-2 is ranged from 400 to 5000, and a number average molecular weight of the structural unit represented by formula I-3 is ranged from 400 to 5000.

A method for preparing a polyurethane elastomer, includes:
copolymerizing the raw material mixture for the polyurethane elastomer to achieve the polyurethane elastomer.

A polyurethane elastomer is prepared by the above method, including copolymerizing the raw material mixture for the polyurethane elastomer.

A raw material composition for polyurethane-modified silicone rubber, includes raw materials for silicone rubber, a raw material mixture for the polyurethane elastomer, and a compatibilizer; the raw materials for silicone rubber include a first silicone rubber component C1 and a second silicone rubber component C2; wherein:
a weight ratio of the first silicone rubber component C1 to the raw material mixture for the polyurethane elastomer is ranged from 10:1.25 to 10:25;
a weight ratio of the first silicone rubber component C1 to the compatibilizer is ranged from 10:0.125 to 10:7.5;
a weight ratio of the first silicone rubber component C1 to the second silicone rubber component C2 is ranged from 10:0.5 to 10:5;
the first silicone rubber component C1 includes vinyl silicone rubber and a crosslinking agent, wherein the structure of the vinyl silicone rubber is represented by formula VI, and R₁ₐ, R_{1b}, R_{1c}, R_{1d} are each independently selected from H, substituted or unsubstituted C1-C5 linear or branched alkyl groups, substituted or unsubstituted C6-C20 aromatic groups, n2≥1000;

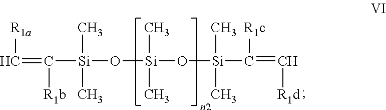

the crosslinking agent contains a silicon-hydrogen bond;
the second silicone rubber component C2 includes a catalyst capable of catalyzing an addition reaction between the vinyl silicone rubber represented by formula VI and the crosslinking agent.

A method for preparing polyurethane-modified silicone rubber, includes:
mixing components of the raw material composition for the polyurethane-modified silicone rubber and curing the mixture to obtain a shaped product.

A polyurethane-modified silicone rubber is prepared by a method including:
mixing raw materials for silicone rubber, the raw material mixture for the polyurethane elastomer, and a compatibilizer to form a mixture; and
curing the mixture to obtain a shaped product;
wherein the raw materials for silicone rubber include a first silicone rubber component C1 and a second silicone rubber component C2, wherein:
a weight ratio of the first silicone rubber component C1 to the raw material mixture for the polyurethane elastomer is ranged from 10:1.25 to 10:25;
a weight ratio of the first silicone rubber component C1 to the compatibilizer is ranged from 10:0.125 to 10:7.5;
a weight ratio of the first silicone rubber component C1 to the second silicone rubber component C2 is ranged from 10:0.5 to 10:5;
the first silicone rubber component C1 includes vinyl silicone rubber and a crosslinking agent, wherein the structure of the vinyl silicone rubber is represented by formula VI, and R₁ₐ, R_{1b}, R_{1c}, R_{1d} are each independently selected from H, substituted or unsubstituted C1-C5 linear or branched alkyl groups, substituted or unsubstituted C6-C20 aromatic groups, n2≥1000;

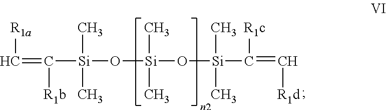

the crosslinking agent contains a silicon-hydrogen bond;
the second silicone rubber component C2 includes a catalyst capable of catalyzing an addition reaction between the vinyl silicone rubber represented by formula VI and the crosslinking agent.

A polyurethane-modified silicone rubber includes:
a silicone rubber base material; and
the above-described polyurethane elastomer, wherein the polyurethane elastomer is dispersed in the silicone rubber base material.

An acoustic lens includes the polyurethane elastomer or the polyurethane-modified silicone rubber.

An ultrasonic probe equipped with the acoustic lens.

### DETAILED DESCRIPTION

In order to make the above objectives, features and advantages of the present application more clear and understandable, embodiments of the present application will be described in detail below with reference to examples. In the following description, many specific details are explained to make the present application fully understandable. However, the present application can be implemented in many other ways different from those described herein, and those skilled in the art can make similar improvements without departing from the connotation of the present application. Therefore, the present application is not limited by the specific embodiments disclosed below.

The present application provides embodiments of a polyurethane elastomer, modified silicone rubber, an acoustic lens, and an ultrasonic probe to solve the problems that in related art there is a difference between the acoustic impedance of an acoustic lens silicone rubber material and the acoustic impedance of the human body and that the acoustic attenuation of the acoustic lens silicone rubber material is relatively high. The polyurethane elastomer-modified silicone rubber in at least some embodiments of the present application can improve the acoustic performance of silicone rubber, specifically can effectively enhance the acoustic impedance of silicone rubber and reduce the acoustic attenuation of silicone rubber, and thus can be used as an acoustic lens material with improved performance.

As a molecule of a polyurethane elastomer has both soft and hard chain segments, through research and development, the inventors found that the shear modulus of the polyurethane elastomer can be changed through molecular chain design, thereby adjusting the acoustic performance of the polyurethane elastomer. Specifically, the first prepolymer component is configured to form the soft chain segments of the polyurethane elastomer, and the second prepolymer component is configured to form the hard chain segments of the polyurethane elastomer. Based on this, the present application provides a method involving molecular chain adjustment to prepare polyurethane elastomers with different acoustic performances and mechanical strengths. These elastomers are then used to modify room temperature vulcanizing silicone rubber through blending and curing, which can effectively enhance the acoustic impedance of silicone rubber and reduce the acoustic attenuation of silicone rubber.

In some specific embodiments, different polyurethane elastomers with adjusted molecular chains are prepared and then used to modify the room temperature vulcanizing silicone rubber RTV630, which improves the acoustic impedance of the silicone rubber while reducing the acoustic attenuation of the silicone rubber.

Some embodiments of the present application provide an acoustic polyurethane elastomer and a preparation method thereof. The acoustic performance of the elastomer is affected by the ratio and types of soft and hard molecular chains in the composition. The polyurethane elastomer is prepared through a semi-prepolymer method involving component type regulation, which can further regulate the acoustic performance of the polyurethane elastomer. The preparation process is stable and feasible.

The present application provides a polyurethane elastomer, which includes a structural unit represented by formula I and a chain extender unit:

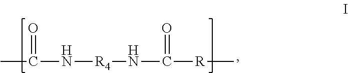

wherein the structural unit represented by formula I is a polyurethane main chain structural unit, which can be a repeating unit, where R₄ is C6-C20 arylene or C6-C20 cycloalkylene, and R is selected from structural units represented by formula I-1 and/or formula I-2 and/or formula I-3:

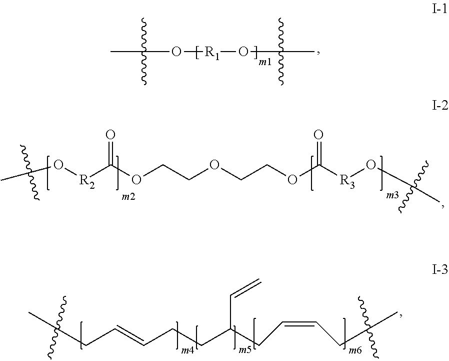

where R₁, R₂, and R₃ are each independently selected from C2-C10 hydrocarbon chains, and m1 to m6 are all positive integers;
a number average molecular weight (Mn) of the structural unit represented by formula I-1 is ranged from 400 to 5000, a number average molecular weight (Mn) of the structural unit represented by formula I-2 is ranged from 400 to 5000, and a number average molecular weight of the structural unit represented by formula I-3 is ranged from 400 to 5000; and
a molar ratio of the chain extender unit to the structural unit represented by formula I is ranged from 0.3:1 to 0.8:1.

In some embodiments, the polyurethane elastomer further includes a structural unit represented by formula II.

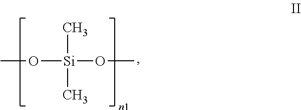

wherein the structural unit represented by formula II is a polysiloxane structural unit, a number average molecular weight (Mn) of the polysiloxane structural unit is ranged from 500 to 6000, and n1 is a positive integer;
a molar ratio of the structural unit represented by formula II to the structural unit represented by formula I is ranged from 0.001:1 to 0.2:1.

In some embodiments, the molar ratio of the structural unit represented by formula II to the structural unit represented by formula I is ranged from 0.005:1 to 0.2:1, such as ranged from 0.02:1 to 0.2:1, ranged from 0.015:1 to 0.12:1, ranged from 0.025:1 to 0.15:1, ranged from 0.005:1 to 0.05:1, ranged from 0.005:1 to 0.06:1, or ranged from 0.025:1 to 0.15:1.

In some embodiments, the number average molecular weight (Mn) of the polysiloxane structural unit is ranged from 500 to 6000, optionally ranged from 1000 to 3000, for example, 2000 or 3000.

In some embodiments, the polyurethane elastomer is a block copolymer consisting of one or more structural units represented by formula I and one or more chain extender units.

In some embodiments, the polyurethane elastomer is a block copolymer consisting of one or more structural units represented by formula I, one or more structural units represented by formula II, and one or more chain extender units.

In some embodiments, a repeating unit of the block copolymer is represented by a formula of:

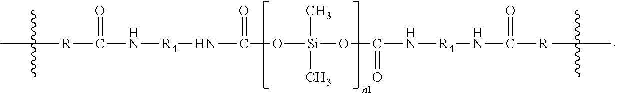

In some embodiments, the hydrocarbon chains of R₁, R₂, and R₃ each include at least one unsaturated bond.

In some embodiments, the number of side chains in each hydrocarbon chain of R₁, R₂, and R₃ is smaller than or equal to 2, for example, 0 or 1.

In some embodiments, the number of carbon atoms in each side chain of the hydrocarbon chains of R₁, R₂, and R₃ is smaller than or equal to 2.

In some embodiments, R₁ is selected from C2-C6 hydrocarbon chains, such as C2-C6 alkylene or C2-C6 alkenylene, for example, —CH₂CH₂CH₂CH₂—, —CH₂CH₂—, —CH(CH₃)CH₂—, —CH₂CH═CHCH₂—.

In some embodiments, R₂ is selected from C2-C6 hydrocarbon chains, such as C2-C6 alkylene, for example, —CH₂CH₂CH₂CH₂CH₂—.

In some embodiments, R₃ is selected from C2-C6 hydrocarbon chains, such as C2-C6 alkylene, for example, —CH₂CH₂CH₂CH₂CH₂—.

In some embodiments, R is the structural unit represented by formula I-1, and R₁ is selected from —CH₂CH₂CH₂CH₂— and —CH(CH₃)CH₂—.

In some embodiments, R is selected from the structural units represented by formula I-1 and formula I-2, R₁ is —CH₂CH₂— or —CH₂CH₂CH₂CH₂—, R₂ is —CH₂CH₂CH₂CH₂CH₂—, and R₃ is —CH₂CH₂CH₂CH₂CH₂—.

In some embodiments, R is the structural unit represented by formula I-1, R₁ is selected from —CH(CH₃)CH₂— and —CH₂CH═CHCH₂—.

In some embodiments, R is the structural unit represented by formula I-2, R₂ is —CH₂CH₂CH₂CH₂CH₂—, and R₃ is —CH₂CH₂CH₂CH₂CH₂—.

In some embodiments, R is the structural unit represented by formula I-1, R₁ is selected from —CH₂CH₂— and —CH₂CH═CHCH₂—.

In some embodiments, R₄ is selected from the following structures and any combination thereof:

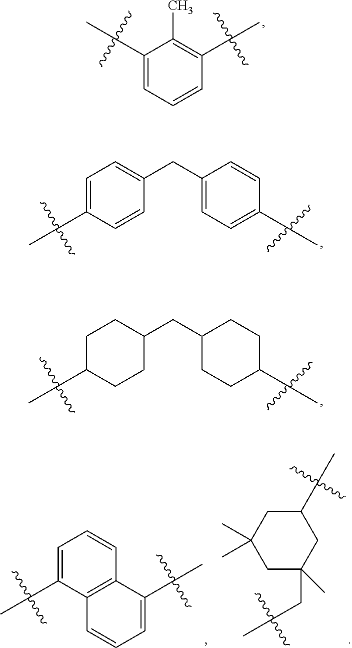

In some embodiments, the chain extender unit is a structural unit represented by

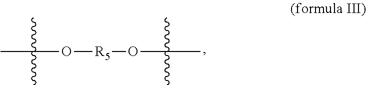

where R₅ is selected from C3-C10 hydrocarbon chains.

In some embodiments, R₅ is selected from C2-C6 hydrocarbon chains, such as C2-C6 alkylene, for example, —CH₂CH₂CH₂CH₂—, —CH₂CH₂CH₂CH₂CH₂CH₂—, —CH₂CH(OH)CH₂—, —CH₂CH(OH)CH(OH)CH(OH)CH(OH)CH₂—. —CH₂(CH₂CH₃)C(CH₂OH)CH₂—.

In some embodiments, a molar ratio of the chain extender unit to the structural unit represented by formula I is ranged from 0.3:1 to 0.6:1, for example, ranged from 0.45:1 to 0.6:1, and in some embodiments is 0.3:1, 0.4:1, 0.5:1, or 0.6:1.

In some embodiments, the sound velocity of the polyurethane elastomer is ranged from 1500 m/s to 1650 m/s, for example, 1541 m/s, 1573 m/s, 1608 m/s, 1517 m/s, 1535 m/s, 1588 m/s, or 1541 m/s.

In some embodiments, the acoustic impedance of the polyurethane elastomer is ranged from 1.50 MRayl to 1.70 MRayl, for example, 1.59 MRayl, 1.65 MRayl, 1.58 MRayl, 1.61 MRayl, or 1.60 MRayl.

In the present application, the acoustic impedance of the polyurethane elastomer can be obtained using an oscilloscope through underwater acoustic measurement. For example, first, the density of the sample is calculated using the simple density formula: ρ=m/V, where m is the mass of the sample and V is the volume of the sample. Next, the sound velocity and the acoustic attenuation coefficient of the material can be obtained through the water immersion method according to standard practice ASTM E664-93 (2000), wherein a first test sample and a second test sample made of the same material and having different thicknesses are alternately inserted into water, and sound is transmitted through the water. The sound propagation time in the water is recorded. The acoustic impedance of the material can be calculated as follows:

$$C = ( {l}_{{1}^{-}} {l}_{2} ) {C}_{0} / ( Δ {tC}_{0} + ( {l}_{{1}^{-}} {l}_{2} ) ) ;$$

$$α = ( 20 lg ( {A}_{1} / {A}_{2} ) ) / ( {l}_{{1}^{-}} {l}_{2} ) + {α}_{0} ;$$

$$Z = ρ C ;$$

wherein C represents the sound velocity of the test sample material, I₁ represents the thickness of the first test sample, I₂ represents the thickness of the second test sample, Δt represents the sound propagation time difference between the water with the first test sample inserted and the water with the second test sample inserted, C₀ is the sound velocity in water, α₀ is the acoustic attenuation coefficient in water, A₁ and A₂ are the acoustic pulse signal amplitudes received when the first and second test samples were inserted in the water respectively, α is the acoustic attenuation coefficient of the test sample material in water, and Z is the acoustic impedance of the test sample material.

In some embodiments, with respect to a sound with a frequency of 2 MHz, an acoustic attenuation of the polyurethane elastomer is ranged from 4.00 dB/cm to 8.00 dB/cm, for example 6.55 dB/cm, 7.57 dB/cm, 6.08 dB/cm, 5.79 dB/cm, 4.98 dB/cm, 6.98 dB/cm, or 6.67 dB/cm.

In some embodiments, a hardness of the polyurethane elastomer is ranged from 40 HA to 70 HA, for example, 60 HA, 55 HA, 45 HA, 40 HA, or 65 HA.

In the present application, the hardness of the polyurethane elastomer can be measured using a Shore hardness tester, and the measuring method can be referred to the GB/T531.1-2008 standard.

The present application further provides a raw material mixture for preparing the polyurethane elastomer, which includes a first prepolymer component and a second prepolymer component.

The first prepolymer component includes a first raw material A, a catalyst, and a chain extender with a chain extension coefficient ranged from 0.3 to 0.8;
the first prepolymer component and the second prepolymer component satisfy an isocyanate index R ranged from 0.8 to 1.2;
where:
the first raw material A includes a raw material of formula IV-1, formula IV-2, or formula IV-3:

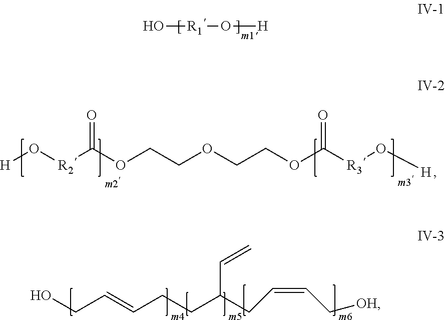

where R₁′, R₂′, and R₃′ are each independently selected from C2-C10 hydrocarbon chains, and m1′, m2′, m3′, m4, m5, and m6 are all positive integers;
a number average molecular weight (Mn) of the raw material of formula IV-1 or formula IV-2 or formula IV-3 is ranged from 400 to 5000;
the structure of the second prepolymer component is represented by formula V:

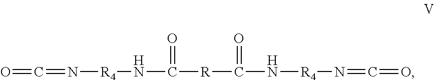

where R₄ is C6-C20 arylene or C6-C20 cycloalkylene, and R is selected from structural units represented by formula I-1 and/or formula I-2 and/or formula I-3:

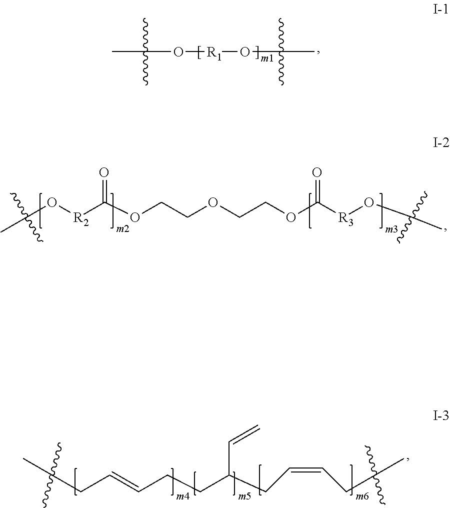

where R₁, R₂, and R₃ are each independently selected from C2-C10 hydrocarbon chains, and m1, m2, m3, m4, m5, and m6 are all positive integers;
a number average molecular weight (Mn) of the structural unit represented by formula I-1 is ranged from 400 to 5000, a number average molecular weight (Mn) of the structural unit represented by formula I-2 is ranged from 400 to 5000, and a number average molecular weight of the structural unit represented by formula I-3 is ranged from 400 to 5000.

In some embodiments, a water content of the first raw material A (e.g., an oligomeric polyol or an alkylene oxide) is generally less than 0.05 wt %, where “wt %” refers to the mass percentage in the first raw material A.

In some embodiments, the first raw material A takes 60 to 90 parts by mass, for example, 65 to 80 parts by mass, further for example, 70 to 80 parts by mass, and still further for example, 65, 70, 75, or 80 parts by mass.

In some embodiments, the hydrocarbon chains of R₁′, R₂′, and R₃′ each include at least one unsaturated bond.

In some embodiments, the number of side chains in each hydrocarbon chain of R₁′, R₂′, and R₃′ is smaller than or equal to 2, for example, 0 or 1.

In some embodiments, the number of carbon atoms in each side chain of the hydrocarbon chains of R₁′, R₂′, and R₃′ is smaller than or equal to 2.

In some embodiments, R₁′ is selected from C2-C6 hydrocarbon chains, such as C2-C6 alkylene or C2-C6 alkenylene, for example, —CH₂CH₂CH₂CH₂—, —CH₂CH₂—, —CH(CH₃)CH₂—, —CH₂CH═CHCH₂—.

In some embodiments, R₂′ is selected from C2-C6 hydrocarbon chains, such as C2-C6 alkylene, for example, —CH₂CH₂CH₂CH₂CH₂—.

In some embodiments, R₃′ is selected from C2-C6 hydrocarbon chains, such as C2-C6 alkylene, for example, —CH₂CH₂CH₂CH₂CH₂—.

In some embodiments, R₁′ is —CH₂CH₂CH₂CH₂— or —CH₂CH₂—, R₂′ is —CH₂CH₂CH₂CH₂CH₂—, and R₃′ is —CH₂CH₂CH₂CH₂CH₂—.

In some embodiments, R₁′ is —CH(CH₃)CH₂— or —CH₂CH═CHCH₂—.

In some embodiments, R₂′ is —CH₂CH₂CH₂CH₂CH₂—, and R₃′ is —CH₂CH₂CH₂CH₂CH₂—.

In some embodiments, R₁′ is —CH₂CH₂— or —CH₂CH═CHCH₂—.

In some embodiments, the raw material of formula IV-1 or formula IV-2 is an oligomeric polyol or an alkylene oxide.

In some embodiments, the raw material of formula IV-1 or formula IV-2 is one or more selected from the following materials:
polytetrahydrofuran diol represented by

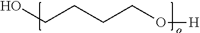

(where o is a positive integer), polycaprolactone diol represented by

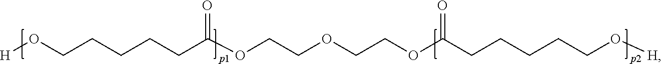

polyethylene glycol represented by

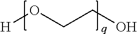

(where q is a positive integer), polypropylene glycol represented by

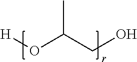

(where r is a positive integer).

In some embodiments, the raw material of formula IV-3 is hydroxyl-terminated polybutadiene.

In some embodiments, the number average molecular weight (Mn) of the raw material of formula IV-1 or formula IV-2 is ranged from 500 to 4000, for example, ranged from 1000 to 3000, further for example, 1000, 1500, 2000, or 3000.

In some embodiments, a number average molecular weight (Mn) of the hydroxyl-terminated polybutadiene is ranged from 500 to 4000, and can be further ranged from 500 to 3000, for example, 1000.

In some embodiments, the raw material of formula IV-1 or formula IV-2 is one or more selected from the following materials:

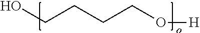

polytetrahydrofuran diol represented by (where o is a positive integer) with a number average molecular weight (Mn) ranged from 500 to 3000, polycaprolactone diol represented by

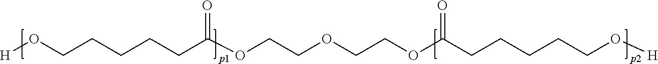

(where p1 and p2 are positive integers) with a number average molecular weight (Mn) ranged from 500 to 3000, polyethylene glycol represented by

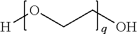

(where q is a positive integer) with a number average molecular weight (Mn) ranged from 400 to 2000, and polypropylene glycol represented by

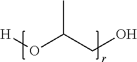

(where r is a positive integer) with a number average molecular weight (Mn) ranged from 1000 to 4000.

In the present application, o, p1, p2, q, and r are generally positive integers; their specific values can be determined based on the number average molecular weight (Mn) of the raw material.

In some embodiments, the raw material of formula IV-1 or formula IV-2 is one or more selected from the following materials: polytetrahydrofuran diol with a number average molecular weight (Mn) ranged from 1000 to 3000, polycaprolactone diol with a number average molecular weight (Mn) ranged from 1000 to 2000, polypropylene glycol with a number average molecular weight (Mn) ranged from 1000 to 1500.

In some embodiments, the raw material of formula IV-3 is hydroxyl-terminated polybutadiene represented by HO—[CH₂CH═CHCH₂]ᵣ—OH (where r is a positive integer) with a number average molecular weight (Mn) ranged from 1000 to 3000, for example, hydroxyl-terminated polybutadiene with a number average molecular weight (Mn) ranged from 1000 to 2000.

In some embodiments, the first prepolymer component further includes a hydroxyl-terminated polysiloxane.

In some embodiments, the structure of the hydroxyl-terminated polysiloxane is represented by the following formula:

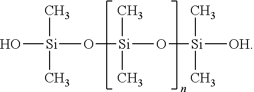

In some embodiments, the number average molecular weight (Mn) of the hydroxyl-terminated polysiloxane is ranged from 500 to 6000.

In some embodiments, the hydroxyl-terminated polysiloxane takes 1.0 to 10.0 parts by mass, for example, 2.0 to 8.0 parts by mass, further for example, 2.0 to 7.0 parts by mass, such as 4.8, 5.0, 5.5, 6.5, 7.5, or 8.0 parts by mass.

In some embodiments, the number average molecular weight of the hydroxyl-terminated polysiloxane is ranged from 500 to 6000, optionally ranged from 1000 to 3000, for example, 2000 or 3000.

In some embodiments, the catalyst takes 0.2 to 1.2 parts by mass, for example, 0.3 to 0.7 parts by mass, further for example, 0.3 to 0.6 parts by mass, and such as 0.32 parts by mass, 0.35 parts by mass, 0.40 parts by mass, 0.45 parts by mass, or 0.60 parts by mass.

In the present application, the catalyst is capable of catalyzing the reaction between the first raw material A (e.g., a polyol or an alkylene oxide) and an isocyanate group, essentially catalyzing the reaction between —OH and —NCO.

In some embodiments, the catalyst is selected from an organotin catalyst and/or an organobismuth catalyst.

The organotin catalyst can be selected from dibutyltin dilaurate, stannous octoate, dibutyltin diacetate, and combinations thereof, for example, dibutyltin dilaurate or stannous octoate.

The organobismuth catalyst can be selected from bismuth octoate, bismuth laurate, and bismuth neodecanoate, and combinations thereof, for example, bismuth octoate.

In some embodiments, the raw material mixture further includes a defoamer.

In some embodiments, the defoamer takes 0.5 to 2.0 parts by mass, for example, 0.8 to 1.2 parts by mass, and further for example, 1.0 part by mass or 1.2 parts by mass.

In some embodiments, the defoamer is selected from a silicone-based defoamer, a mineral oil-based defoamer, and a polymer-based defoamer, and combinations thereof, and optionally is a silicone-based defoamer.

In some embodiments, the defoamer is selected from BYK-A500, BYK-A501, BYK-A515, and combinations thereof, for example, BYK-A500, BYK-A501, or BYK-A515.

In the present application, the chain extender generally refers to a reagent containing an active hydrogen group.

In some embodiments, the chain extension coefficient of the chain extender is ranged from 0.3 to 0.6, for example, ranged from 0.45 to 0.6, such as 0.3, 0.4, 0.5, or 0.6.

In the present application, the chain extension coefficient refers to the ratio of the total molar amount of amino and hydroxyl groups in the chain extender to the molar amount of isocyanate groups in the prepolymer, i.e., the molar ratio of active hydrogen groups to NCO groups.

In some embodiments, the chain extender is an amine-based chain extender.

In some embodiments, the chain extender is a polyol chain extender, such as C3-C10 polyol, for example, C3-C6 polyol, and can be selected from 1,4-butanediol (BDO,

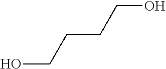

), 1,6-hexanediol (

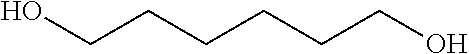

), glycerol (

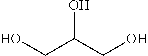

), trimethylolpropane (

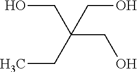

), sorbitol (

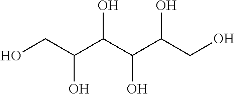 [SMILES: OCC(O)C(O)C(O)C(O)CO]

), and combinations thereof, such as 1,4-butanediol (BDO), 1,6-hexanediol, glycerol, or sorbitol.

In some embodiments, 60 to 90 parts by mass of the first raw material A, 1.0 to 10.0 parts by mass of the hydroxyl-terminated polysiloxane, 0.2 to 1.2 parts by mass of the catalyst, and the chain extender with the chain extension coefficient ranged from 0.3 to 0.8 are mixed uniformly to obtain the first prepolymer component. The first prepolymer component is then mixed with the second prepolymer component to obtain the raw material mixture.

In the present application, the sub-components of the first prepolymer component are physically mixed without involving any chemical reaction.

In some embodiments, the first prepolymer component is further dehydrated before mixing with the second prepolymer component.

The dehydration is performed at a temperature ranged from 70° C. to 120° C., for example, 110° C.

The dehydration can be performed under vacuum, for example, at a vacuum degree of −0.095 MPa.

The dehydration can be performed until the water content is reduced to below 0.05 wt %, where “wt %” refers to the mass percentage in the first prepolymer component.

The dehydration time can be ranged from 1 hour to 10 hours, for example, 1.5 hours or 2 hours.

In some embodiments, the first prepolymer component is mixed under stirring. The stirring speed can be conventional for the field, capable of dispersing the raw materials within the first prepolymer component, for example, ranged from 350 rpm to 500 rpm.

In some embodiments, the second prepolymer component is prepared using the following method:

40 to 70 parts by mass of a second raw material B and 25 to 50 parts by mass of an diisocyanate are subjected to a polymerization reaction to obtain the second prepolymer component; the second raw material B includes a raw material of formula IV-1 or formula IV-2:

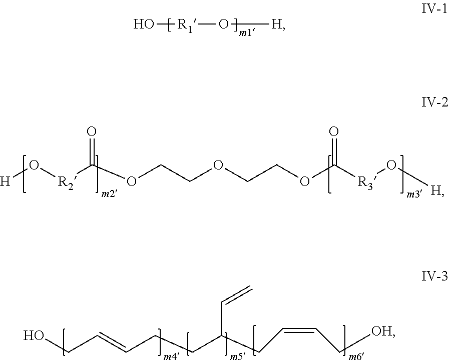

where R₁′, R₂′, and R₃′ are each independently selected from C2-C10 hydrocarbon chains, and m1′, m2′, m3′, m4′, m5′, and m6′ are all positive integers.

In the present application, in the preparation method of the second prepolymer component, the water content of the second raw material B (e.g., an oligomeric polyol or an alkylene oxide) is generally less than 0.05 wt %, where “wt %” refers to the mass percentage in the second raw material B.

In the preparation method of the second prepolymer component of the present application, due to the hygroscopic nature of the second raw material B (e.g., an oligomeric polyol or an alkylene oxide) and the vigorous reaction between water and isocyanates, dehydration is generally performed before the polymerization reaction.

In some embodiments, in the preparation method of the second prepolymer component, the second raw material B takes 50 to 70 parts by mass, for example, 50 to 60 parts by mass, such as 55, 57, 60, or 65 parts by mass.

In some embodiments, in the preparation method of the second prepolymer component, the polymerization reaction is performed at a temperature ranged from 70° C. to 90° C., for example, 80° C., 85° C., or 90° C.

In some embodiments, in the preparation method of the second prepolymer component, the polymerization reaction time is ranged from 1 hour to 10 hours, for example, ranged from 2 hours to 4 hours, such as 1 hour, 1.5 hours, 2.0 hours, or 2.5 hours.

In some embodiments, in the preparation method of the second prepolymer component, the polymerization reaction is terminated when the NCO content is ranged from 7 wt % to 20 wt %, for example, 10 wt % to 15 wt %, such as 14.7 wt %, 11.75 wt %, 8.8 wt %, 9.5 wt %, or 15.85 wt %, where “wt %” refers to the mass percentage in the second prepolymer component.

In some embodiments, the structure of R₁′ in the first raw material A is identical to the structure of R₁ in the second prepolymer component.

In some embodiments, the structure of R₁′ in the first raw material A is different from the structure of R₁ in the second prepolymer component.

In some embodiments, the structure of R₂′ in the first raw material A is identical to the structure of R₂ in the second prepolymer component.

In some embodiments, the structure of R₂′ in the first raw material A is different from the structure of R₂ in the second prepolymer component.

In some embodiments, the structure of R₃′ in the first raw material A is identical to the structure of R₃ in the second prepolymer component.

In some embodiments, the structure of R₃′ in the first raw material A is different from the structure of R₃ in the second prepolymer component.

In some embodiments, a mass ratio of the first raw material A in the first prepolymer component to the second raw material B in the second prepolymer component is ranged from 1:1 to 1.5:1, for example, 1.36:1, 1.23:1, 1.25:1, 1.45:1, or 1.18:1.

In some embodiments, the diisocyanate takes 30 to 50 parts by mass, for example, 30 to 40 parts by mass, such as 38, 40, or 45 parts by mass.

In some embodiments, the diisocyanate is OCN—R₄—NCO, where R₄ is selected from C6-C20 aryl or cycloalkyl groups.

In some embodiments, R₄ is selected from the following structures and any combination thereof:

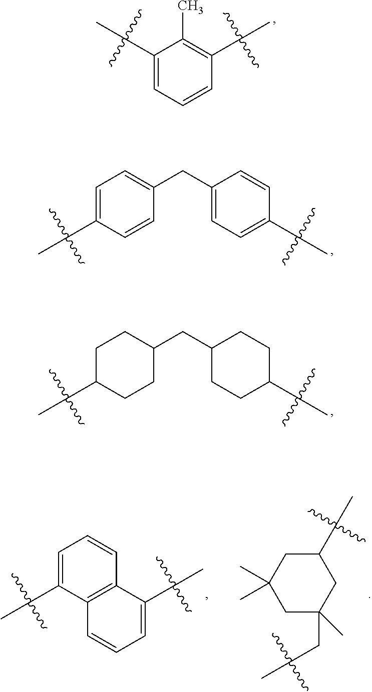

In some embodiments, the diisocyanate is selected from toluene diisocyanate (TDI,

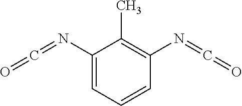

), methylene diphenyl diisocyanate (MDI,

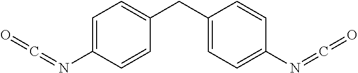 [SMILES: O=C=Nc1ccc(Cc2ccc(N=C=O)cc2)cc1]

), dicyclohexylmethane diisocyanate (HMDI,

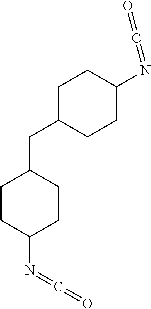 [SMILES: O=C=NC1CCC(CC2CCC(N=C=O)CC2)CC1]

), 1,5-naphthalene diisocyanate (NDI,

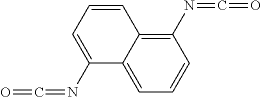 [SMILES: O=C=Nc1cccc2c(N=C=O)cccc12]

), isophorone diisocyanate (IPDI,

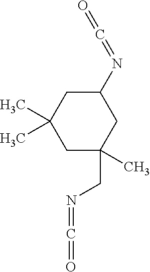

), p-xylylene diisocyanate (XDI,

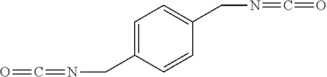 [SMILES: O=C=NCc1ccc(CN=C=O)cc1]

), and any combination thereof, and optionally is dicyclohexylmethane diisocyanate (HMDI) or methylene diphenyl diisocyanate (MDI).

In some embodiments, the hydrocarbon chains of R₁, R₂, and R₃ each include at least one unsaturated bond.

In some embodiments, the number of side chains in each hydrocarbon chain of R₁, R₂, and R₃ is smaller than or equal to 2, for example, 0 or 1.

In some embodiments, the number of carbon atoms in each side chain of the hydrocarbon chains of R₁, R₂, and R₃ is smaller than or equal to 2.

In some embodiments, R₁ is selected from C2-C6 hydrocarbon chains, such as C2-C6 alkylene or C2-C6 alkenylene, for example, —CH₂CH₂CH₂CH₂—, —CH₂CH₂—, —CH(CH₃)CH₂—, —CH₂CH═CHCH₂—.

In some embodiments, R₂ is selected from C2-C6 hydrocarbon chains, such as C2-C6 alkylene, for example, —CH₂CH₂CH₂CH₂CH₂—.

In some embodiments, R₃ is selected from C2-C6 hydrocarbon chains, such as C2-C6 alkylene, for example, —CH₂CH₂CH₂CH₂CH₂—.

In some embodiments, R is selected from the structural units represented by formula I-1 and formula I-2, R₁ is —CH₂CH₂— or —CH₂CH₂CH₂CH₂—, R₂ is —CH₂CH₂CH₂CH₂CH₂—, and R₃ is —CH₂CH₂CH₂CH₂CH₂—.

In some embodiments, R is the structural unit represented by formula I-1, R₁ is selected from —CH(CH₃)CH₂— and —CH₂CH—CHCH₂—.

In some embodiments, R is the structural unit represented by formula I-2, R₂ is —CH₂CH₂CH₂CH₂CH₂—, and R₃ is —CH₂CH₂CH₂CH₂CH₂—.

In some embodiments, R is the structural unit represented by formula I-1, R₁ is selected from —CH₂CH₂— and —CH₂CH═CHCH₂—.

In the present application, isocyanate index R refers to the molar ratio of NCO groups to active hydrogen groups (e.g., hydroxyl and amino groups) in the reaction system.

In some embodiments, the first prepolymer component and the second prepolymer component satisfy the isocyanate index R=0.9 to 1.1, for example I=0.95.

In some embodiments, a mass ratio of the first prepolymer component to the second prepolymer component is ranged from 10:1 to 10:10, for example 10:6, 10:7.5, 10:4.0, 10:3.3, 10:4.5, or 10:10.

The present application further provides a method for preparing the polyurethane elastomer, which includes the following step:
copolymerizing the raw material mixture for the polyurethane elastomer to achieve the polyurethane elastomer.

In some embodiments, the copolymerization condition of the raw material mixture for the polyurethane elastomer is room temperature curing, e.g., curing at 25° C. for 24 hours.

In some embodiments, before the copolymerization, the raw material mixture for the polyurethane elastomer is mixed at a high speed (e.g., 350 to 500 rpm) for 1 to 3 minutes and degassed under vacuum.

The present application further provides a polyurethane elastomer prepared by the above method.

The present application further provides a raw material composition for polyurethane-modified silicone rubber, which includes raw materials for silicone rubber, the raw material mixture for the polyurethane elastomer, and a compatibilizer. The raw materials for silicone rubber include a first silicone rubber component C1 and a second silicone rubber component C2; wherein:
a weight ratio of the first silicone rubber component C1 to the raw material mixture for the polyurethane elastomer is ranged from 10:1.25 to 10:25;
a weight ratio of the first silicone rubber component C1 to the compatibilizer is ranged from 10:0.125 to 10:7.5;
a weight ratio of the first silicone rubber component C1 to the second silicone rubber component C2 is ranged from 10:0.5 to 10:5;
the first silicone rubber component C1 includes vinyl silicone rubber and a crosslinking agent, wherein the structure of the vinyl silicone rubber is represented by formula VI, and R₁ₐ, R_{1b}, R_{1c}, R_{1d} are each independently selected from H, substituted or unsubstituted C1-C5 linear or branched alkyl groups, substituted or unsubstituted C6-C20 aromatic groups, n2≥1000;

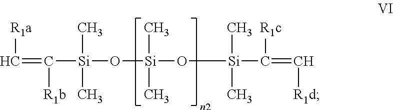

the crosslinking agent contains a silicon-hydrogen bond;
the second silicone rubber component C2 includes a catalyst capable of catalyzing an addition reaction between the vinyl silicone rubber represented by formula VI and the crosslinking agent.

In some embodiments, the weight ratio of the first silicone rubber component C1 to the second silicone rubber component C2 is 10:1.

In some embodiments, in the raw material mixture for the polyurethane elastomer, the first prepolymer component and the second prepolymer component are proportioned according to isocyanate index R=0.8 to 1.2, for example, R=0.9 to 1.1; further for example, the first prepolymer component and the second prepolymer component are proportioned according to isocyanate index R=0.95.

In some embodiments, in the raw material mixture for the polyurethane elastomer, the first prepolymer component can include other sub-components except the second prepolymer component. For example, the first prepolymer component includes: 60 to 90 parts by mass of the first raw material A, 1.0 to 10.0 parts by mass of the hydroxyl-terminated polysiloxane, 0.2 to 1.2 parts by mass of the catalyst, and the chain extender with a chain extension coefficient ranged from 0.3 to 0.8.

In some embodiments, the weight ratio of the first silicone rubber component C1 to the raw material mixture for the polyurethane elastomer is ranged from 10:2.0 to 10:20.0, for example, 10:5.60, 10:8.75, 10:7.98, 10:7.25, or 10:18.00.

In some embodiments, the weight ratio of the first silicone rubber component C1 to the first prepolymer component in the raw material mixture for the polyurethane elastomer is ranged from 10:3.5 to 10:10, for example, 10:3.5, 10:4.0, 10:5.0, 10:6.0, or 10:10.0.

In some embodiments, the weight ratio of the first silicone rubber component C1 to the compatibilizer is ranged from 10:0.5 to 10:5, for example, 10:0.6, 10:0.75, or 10:1.5.

In some embodiments, the compatibilizer is a silane coupling agent.

In some embodiments, the silane coupling agent includes, but is not limited to, KH550, KH560, KH580, or combinations thereof, for example, KH550 or KH560.

In some embodiments, a weight ratio of the first silicone rubber component C1 to the second prepolymer component in the raw material mixture for the polyurethane elastomer is ranged from 10:0.5 to 10:10, for example, 10:1.6, 10:1.98, 10:2.1, 10:2.25, 10:3.75, or 10:8.0.

In some embodiments, in the structure represented by formula VI, R₁ₐ is H.

In some embodiments, in the structure represented by formula VI, R_{1b} is H.

In some embodiments, in the structure represented by formula VI, R_{1c} is H.

In some embodiments, in the structure represented by formula VI, R_{1d} is H.

In some embodiments, in the structure represented by formula VI, R₁ₐ, R_{1b}, R_{1c}, R_{1d} are each H, and the structure of the vinyl silicone rubber is represented by formula VI′.

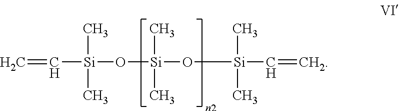

In some embodiments, in the structure represented by formula VI, n2 is ranged from 1000 to 5000, for example 2000.

In some embodiments, a number average molecular weight (Mn) of the vinyl silicone rubber is ranged from 1,000 to 200,000.

In some embodiments, in the first silicone rubber component C1, the crosslinking agent is R≡SiH. The second silicone rubber component C2 can catalyze the addition reaction of R≡SiH in the first silicone rubber component C1.

In some embodiments, the crosslinking agent can be poly(methylhydrosiloxane). The structural formula of poly(methylhydrosiloxane) can be R≡SiH, where R can be an alkyl chain.

In some embodiments, the first silicone rubber component C1 can further includes conventional additives, such as an inhibitor and/or filler. Generally, conventional additives have minimal impact on the main structure of the silicone rubber.

The inhibitor can be a conventional compound capable of inhibiting the addition reaction between the structure of formula VI and the crosslinking agent in the field, such as one or more of an alkynol, a nitrogen-containing compound, or an organic peroxide, for example methylbutynol, or 2-methyl-3-butyn-2-ol.

The filler can be a conventional filler in the field, such as one or more of precipitated silica, titanium dioxide, quartz powder, alumina, zinc oxide, or tungsten oxide, for example precipitated silica.

In the present application, the second silicone rubber component C2 generally includes at least one of a transition metal of Group VIII of the periodic table, or a compound thereof, or a complex thereof, such as platinum, a platinum-containing compound, or a platinum-containing complex.

In the second silicone rubber component C2, the transition metal containing compound or complex in the second silicone rubber component C2 is generally used as a catalyst or a curing agent and typically not as a filler in the silicone rubber matrix.

In some embodiments, the first silicone rubber component C1 can further include one or more of methyl silicone oil, vinyl silicone oil, hydroxyl silicone oil, hydroxymethyl fluoro silicone oil, or end-capped epoxy silicone oil, for example vinyl silicone oil.

In the present application, the second silicone rubber component C2 is capable of catalyzing the addition reaction of —SiCH═CH₂ in the first silicone rubber component C1.

In some embodiments, the raw materials for silicone rubber can be two-component addition-type raw materials for liquid silicone rubber. The two-component addition-type raw materials refer to two components that form silicone rubber through an addition reaction.

In some embodiments, the silicone rubber formed from the silicone rubber raw materials is AB two-component addition-type liquid silicone rubber, for example RTV630 or RTV615 produced by Momentive Inc. RTV630 and RTV615 are AB two-component room temperature vulcanizing (RTV) liquid silicone rubbers.

The AB two-component addition-type liquid silicone rubber is typically cured at room temperature or an elevated temperature (e.g., 30° C. to 100° C., or specifically 50° C. to 70° C.) with a polyorganosiloxane containing Si—H bonds as a crosslinking agent under an action of a catalyst (such as platinum catalyst). The main structure includes a polydiorganosiloxane with two or more vinyl groups (e.g., as shown in formula VI′).

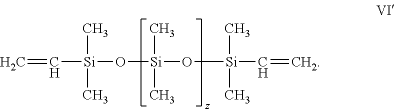

When the first silicone rubber component C1 contains vinyl and silicon-hydrogen bonds, the curing mechanism of the AB two-component addition-type liquid silicone rubber involves the addition reaction of vinyl and silicon-hydrogen bonds catalyzed by a catalyst (such as a platinum compound), hence called as AB two-component addition-type liquid silicone rubber. This rubber can be cured by uniformly mixing the first and second silicone rubber components C1, C2 in a certain ratio and resting the mixture either at room temperature or an elevated temperature (e.g., 30° C. to 100° C., or specifically 50° C. to 70° C.) for a period of time.

Specifically, when the AB two-component addition-type liquid silicone rubber is room temperature vulcanizing silicone rubber RTV630 or RTV615 produced by Momentive Inc, during usage, the two components can be mixed in a ratio of 10:1 by weight (silicone rubber component Al/silicone rubber component B1=10:1), then rest at room temperature or an elevated temperature (e.g., 30° C. to 100° C., or specifically 50° C. to 70° C.) to cure the rubber.

In some embodiments, the silicone rubber is the room temperature vulcanizing silicone rubber RTV630 (the structure is represented by formula VII) produced by Momentive Inc. This two-component silicone rubber compound can be cured into high-strength silicone rubber at room temperature. The product is provided as a matching kit of a base component (A) and a curing agent (B), where the weight ratio of A to B can be 10:1.

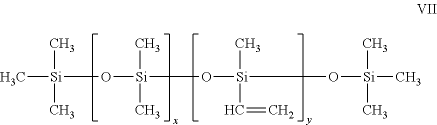

In some embodiments, the raw material composition for polyurethane-modified silicone rubber is any one of compositions 1 to 7, and the components thereof takes following parts by mass.

| | Silicone rubber | Component | Component | First prepolymer | Second prepolymer | |
|---|---|---|---|---|---|---|
| No | type | C1 | C2 | component | component | KH560 |
| 1 | RTV630 | 10 | 1 | 3.5 | 2.10 | 0.6 |
| 2 | RTV630 | 10 | 1 | 5.0 | 3.75 | 0.6 |
| 3 | RTV630 | 10 | 1 | 4.0 | 1.60 | 0.75 |
| 4 | RTV630 | 10 | 1 | 6.0 | 1.98 | 0.75 |
| 5 | RTV630 | 10 | 1 | 5.0 | 2.25 | 0.75 |
| 6 | RTV630 | 10 | 1 | 10.0 | 8.00 | 1.5 |
| 7 | RTV630 | 10 | 1 | 3.5 | 2.1 | 0.6 |

The present application further provides a method for preparing polyurethane-modified silicone rubber, including the following step:
mixing the components of the raw material composition for the polyurethane-modified silicone rubber and curing the mixture to obtain a shaped product.

The curing temperature can be decided based on the properties of the components of the raw material composition for the polyurethane-modified silicone rubber. For example, when the components in the raw material composition can be cured at room temperature, the curing temperature can be room temperature. Alternatively, the curing temperature can be ranged from 20° C. to 100° C., or specifically 25° C. to 40° C.

The curing time can also be decided based on the properties of the components of the raw material composition for the polyurethane-modified silicone rubber, generally ensuring complete reaction. For example, the curing time can be ranged from 12 hours to 60 hours, or specifically 24 hours.

When the raw material composition for the polyurethane-modified silicone rubber is in liquid form, curing can be conducted in a mold.

Before curing, and after mixing, a conventional degassing treatment can be carried out according to a conventional method in the field.

In some embodiments, the method for preparing polyurethane-modified silicone rubber includes the following steps:
(1) mixing the first silicone rubber component C1, the first prepolymer component, and the compatibilizer to obtain a first mixture;
(2) mixing the first mixture with the second silicone rubber component C2 and the second prepolymer component to obtain a second mixture; and curing the second mixture to obtain a shaped product.

In some embodiments, the mixing time for the first mixing is ranged from 1 minute to 10 minutes.

In some embodiments, both the first and second mixings are conducted under high-speed conditions, for example, at 350 rpm to 500 rpm.

The present application further provides a polyurethane-modified silicone rubber prepared by the above method.

The present application further provides a polyurethane-modified silicone rubber including:
a silicone rubber base material, wherein the silicone rubber base material is polymerized from the first silicone rubber component C1 and the second silicone rubber component C2; and
the above-described polyurethane elastomer, wherein the polyurethane elastomer is dispersed in the silicone rubber base material.

In some embodiments, the structure of the silicone rubber base material can be represented by formula VII.

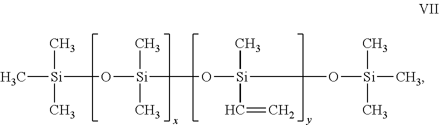

wherein x and y are positive integers.

In some embodiments, the silicone rubber base material is polymerized from room temperature vulcanizing silicone rubber RTV630.

In some embodiments, the sound velocity of the polyurethane-modified silicone rubber is ranged from 1000 m/s to 1500 m/s, for example, 1190 m/s, 1254 m/s, 1218 m/s, 1229 m/s, 1224 m/s, 1386 m/s, or 1202 m/s.

In some embodiments, the acoustic impedance of the polyurethane-modified silicone rubber is ranged from 1.40 MRayl to 1.50 MRayl, for example, 1.45 MRayl, 1.43 MRayl, 1.42 MRayl, 1.41 MRayl, 1.47 MRayl, 1.49 MRayl, or 1.46 MRayl.

In the present application, the acoustic impedance of the polyurethane-modified silicone rubber can be obtained using an oscilloscope through underwater acoustic measurement. For example, first, the density of the sample is calculated using the simple density formula: ρ=m/V, where m is the mass of the sample and V is the volume of the sample. Next, the sound velocity and the acoustic attenuation coefficient of the material can be obtained through the water immersion method according to standard practice ASTM E664-93 (2000), wherein a first test sample and a second test sample made of the same material and having different thicknesses are alternately inserted into water, and sound is transmitted through the water. The sound propagation time in the water is recorded. The acoustic impedance of the material can be calculated as follows:

$$C = ( {l}_{{1}^{-}} {l}_{2} ) {C}_{0} / ( Δ {tC}_{0} + ( {l}_{{1}^{-}} {l}_{2} ) ) ;$$

$$α = ( 20 lg ( {A}_{1} / {A}_{2} ) ) / ( {l}_{{1}^{-}} {l}_{2} ) + {α}_{0} ;$$

$$Z = ρ C ;$$

wherein C represents the sound velocity of the test sample material, I₁ represents the thickness of the first test sample, I₂ represents the thickness of the second test sample, Δt represents the sound propagation time difference between the water with the first test sample inserted and the water with the second test sample inserted, C₀ is the sound velocity in water, α₀ is the acoustic attenuation coefficient in water, A₁ and A₂ are the acoustic pulse signal amplitudes received when the first and second test samples were inserted in the water respectively, α is the acoustic attenuation coefficient of the test sample material in water, and Z is the acoustic impedance of the test sample material.

In some embodiments, with respect to a sound with a frequency of 2 MHz, an acoustic attenuation of the polyurethane-modified silicone rubber is ranged from 5.00 dB/cm to 15.00 dB/cm, for example 8.07 dB/cm, 8.95 dB/cm, 8.70 dB/cm, 8.25 dB/cm, 8.03 dB/cm, 7.97 dB/cm, or 12.95 dB/cm.

In some embodiments, a hardness of the polyurethane-modified silicone rubber is ranged from 40 HA to 70 HA, for example, 60 HA, 58 HA, 55 HA, 45 HA, or 62 HA.

In the present application, the hardness of the polyurethane-modified silicone rubber can be measured using a Shore hardness tester, and the measuring method can be referred to the GB/T531.1-2008 standard.

The present application further provides an application of the polyurethane elastomer or the polyurethane-modified silicone rubber as an acoustically transparent material in an ultrasonic endoscope.

An acoustic lens of the ultrasonic endoscope can be made of the acoustically transparent material.

The present application further provides an acoustic lens including the polyurethane elastomer or the polyurethane-modified silicone rubber.

In the embodiments of the present application, room temperature generally refers to 25° C.±5° C.

The present application further provides an ultrasonic probe equipped with the above-described acoustic lens.

Based on common knowledge in the field, the above conditions can be combined arbitrarily to obtain various embodiments of the present application.

The reagents and raw materials used in the embodiments of the present application are all commercially available.

The positive effects of the embodiments of the present application include:
the present application provides a method for changing the acoustic properties of
the polyurethane elastomer by adjusting molecular chains of the polyurethane elastomer;
the present application provides a method for modifying room temperature vulcanizing silicone rubber using the polyurethane elastomer, resulting in improved acoustic impedance of the modified silicone rubber (closer to the acoustic impedance of the human body, approximately 1.5 MRayl) and reduced sound attenuation;
the modification method of the present application significantly enhances the compatibility between the polyurethane elastomer and the room temperature vulcanizing silicone rubber, effectively preventing phase separation, which is beneficial for improving the yield;
the preparation processes of the present application are simple, the subsequent operations are convenient, the polyurethane-modified silicone rubber can be molded at room temperature and atmospheric pressure, which is conducive to the preparation of acoustic lenses.

The following examples are provided to specifically describe the embodiments of the present application. It should be understood that the examples are for illustration only and not intended to limit the scope of the present application. In the following examples, experimental methods not specifying specific conditions should refer first to the guidance given in the present application, or to the experimental manuals or conventional conditions in the art, or to the conditions recommended by manufacturers, or to the experimental methods known in the art.

In the following examples and comparative examples:
(1) The acoustic impedance of the polyurethane elastomer can be obtained using an oscilloscope through underwater acoustic measurement, wherein the detection methods and calculation formulas of the sound velocity, acoustic impedance, and acoustic attenuation are as follows:

First, the density of the sample is calculated using the simple density formula: ρ=m/V, where m is the mass of the sample and V is the volume of the sample.

Next, the sound velocity and the acoustic attenuation coefficient of the material can be obtained through the water immersion method according to standard practice ASTM E664-93 (2000), wherein a first test sample and a second test sample made of the same material and having different thicknesses are alternately inserted into water, and sound is transmitted through the water. The sound propagation time in the water is recorded. The acoustic impedance of the material can be calculated as follows:

$$C = ( {l}_{{1}^{-}} {l}_{2} ) {C}_{0} / ( Δ {tC}_{0} + ( {l}_{{1}^{-}} {l}_{2} ) ) ;$$

$$α = ( 20 lg ( {A}_{1} / {A}_{2} ) ) / ( {l}_{{1}^{-}} {l}_{2} ) + {α}_{0} ;$$

$$Z = ρ C ;$$

wherein C represents the sound velocity of the test sample material, I₁ represents the thickness of the first test sample, I₂ represents the thickness of the second test sample, Δt represents the sound propagation time difference between the water with the first test sample inserted and the water with the second test sample inserted, C₀ is the sound velocity in water, α₀ is the acoustic attenuation coefficient in water, A₁ and A₂ are the acoustic pulse signal amplitudes received when the first and second test samples were inserted in the water respectively, α is the acoustic attenuation coefficient of the test sample material in water, and Z is the acoustic impedance of the test sample material.
(2) The hardness of the polyurethane elastomer can be measured using a Shore hardness tester, and the measuring method can be referred to the GB/T531.1-2008 standard.
(3) The silicone rubber RTV630 is produced by Momentive Inc., whose commercially available form is separately packaged component Al and component B1. In use of the silicone rubber, the two components are uniformly mixed in a mass ratio of 10:1 (A1/B1=10:1), and then cured at room temperature or an elevated temperature (50° C. to 70° C.) in a mold.

The component A1 includes a vinyl polysiloxane, a polymethyl hydrosiloxane, and precipitated silica, wherein the structure of the vinyl polysiloxane is

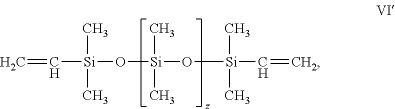

where z=2000; and the component B1 includes platinum catalyst.

According to the technical data sheet (TDS) of RTV630, the Shore hardness of pure RTV630 is 60 HA.
(4) The chain extension coefficient refers to the ratio of the total molar amount of amino and hydroxyl groups in the chain extender to the molar amount of isocyanate groups in the prepolymer, i.e., the molar ratio of active hydrogen groups to NCO groups.
(5) The isocyanate index R refers to the molar ratio of NCO groups to active hydrogen groups (e.g., hydroxyl and amino groups) in the reaction system.
(6) The vacuum degree=atmospheric pressure−absolute pressure.

### Example 1

(1) 75 g of polytetrahydrofuran diol (Mn=1000), 5.5 g of hydroxyl-terminated polysiloxane (Mn=3000), 0.35 g of dibutyltin dilaurate, 1 g of BYK-A501, and 1,4-butanediol (BDO), as a chain extender with a chain extension coefficient of 0.5, were mixed and stirred evenly and then dehydrated at 110° C. and a vacuum degree of −0.095 MPa for 2 hours to obtain a polyurethane prepolymer component A, in which the water content was less than 0.05 wt %.
(2) 55 g of dehydrated (i.e., free water removed) polypropylene glycol (Mn=1500) was reacted with 38 g of dicyclohexylmethane diisocyanate (HMDI) at 80° C. for 2.5 hours to obtain a polyurethane prepolymer component B with NCO %=14.7%.

After that, the polyurethane prepolymer components A and B were mixed in a ratio satisfying the isocyanate index R=0.9 to 1.1. In this example, I=0.95. Specifically, 10 g of the polyurethane prepolymer component A and 6 g of the polyurethane prepolymer component B were mixed at high speed for 2 minutes, then vacuum defoamed, and cured at 25° C. for 24 hours to obtain a polyurethane elastomer with a sound velocity of 1541 m/s, an acoustic impedance of 1.59 Mrayl, an acoustic attenuation of 6.55 dB/cm at 2 MHz, and a Shore A hardness of 60 HA.

In the polyurethane elastomer, the molar ratio of the polysiloxane structural unit to polyurethane main chain structural unit was ranged from about 0.02:1 to about 0.2:1.
(3) 10 g of the component A1 of RTV630, 3.5 g of the polyurethane prepolymer component A prepared in step (1), and 0.6 g of silane coupling agent KH560 were mixed and stirred at high speed for 5 minutes, then 1 g of component B1 of RTV630 and 2.1 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1190 m/s, an acoustic impedance of 1.45 Mrayl, an acoustic attenuation of 8.07 dB/cm at 2 MHz, and a Shore A hardness of 60 HA.

### Example 2

(1) 70 g of polycaprolactone diol (Mn=1000), 4.8 g of hydroxyl-terminated polysiloxane (Mn=2000), 0.4 g of stannous octoate, 1 g of BYK-A515, and sorbitol, as a chain extender with a chain extension coefficient of 0.6, were mixed and stirred evenly and then dehydrated at 110° C. and a vacuum degree of −0.095 MPa for 1.5 hours to obtain a polyurethane prepolymer component A, in which the water content was less than 0.05 wt %.
(2) 57 g of dehydrated (i.e., free water removed) polyethylene glycol (Mn=2000) was reacted with 40 g of methylene diphenyl diisocyanate (MDI) at 85° C. for 1.5 hours to obtain a polyurethane prepolymer component B with NCO %=11.75%.

After that, the polyurethane prepolymer components A and B were mixed in a ratio satisfying the isocyanate index R=0.9 to 1.1. In this example, I=0.95. Specifically, 10 g of the polyurethane prepolymer component A and 7.5 g of the polyurethane prepolymer component B were mixed at high speed for 2 minutes, then vacuum defoamed, and cured at 25° C. for 24 hours to obtain a polyurethane elastomer with a sound velocity of 1573 m/s, an acoustic impedance of 1.60 Mrayl, an acoustic attenuation of 7.57 dB/cm at 2 MHz, and a Shore A hardness of 60 HA.

In the polyurethane elastomer, the molar ratio of the polysiloxane structural unit to polyurethane main chain structural unit was ranged from about 0.015:1 to about 0.12:1.
(3) 10 g of the component A1 of RTV630, 5 g of the polyurethane prepolymer component A prepared in step (1), and 0.6 g of silane coupling agent KH550 were mixed and stirred at high speed for 5 minutes, then 1 g of component B1 of RTV630 and 3.75 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1254 m/s, an acoustic impedance of 1.43 Mrayl, an acoustic attenuation of 8.95 dB/cm at 2 MHZ, and a Shore A hardness of 60 HA.

### Example 3

(1) 75 g of polypropylene glycol (Mn=1500), 7.5 g of hydroxyl-terminated polysiloxane (Mn=3000), 0.6 g of stannous octoate, 1.2 g of BYK-A501, and glycerol, as a chain extender with a chain extension coefficient of 0.5, were mixed and stirred evenly and then dehydrated at 110° C. and a vacuum degree of −0.095 MPa for 2 hours to obtain a polyurethane prepolymer component A, in which the water content was less than 0.05 wt %.
(2) 60 g of dehydrated (i.e., free water removed) hydroxyl-terminated polybutadiene (Mn=1000) was reacted with 45 g of methylene diphenyl diisocyanate (MDI) at 90° C. for 2 hours to obtain a polyurethane prepolymer component B with NCO %=8.8%.

After that, the polyurethane prepolymer components A and B were mixed in a ratio satisfying the isocyanate index R=0.9 to 1.1. In this example, I=0.95. Specifically, 10 g of the polyurethane prepolymer component A and 4 g of the polyurethane prepolymer component B were mixed at high speed for 2 minutes, then vacuum defoamed, and cured at 25° C. for 24 hours to obtain a polyurethane elastomer with a sound velocity of 1608 m/s, an acoustic impedance of 1.65 Mrayl, an acoustic attenuation of 6.08 dB/cm at 2 MHz, and a Shore A hardness of 55 HA.

In the polyurethane elastomer, the molar ratio of the polysiloxane structural unit to polyurethane main chain structural unit was ranged from about 0.025:1 to 0.15:1.
(3) 10 g of the component A1 of RTV630, 4 g of the polyurethane prepolymer component A prepared in step (1), and 0.75 g of silane coupling agent KH550 were mixed and stirred at high speed for 5 minutes, then 1 g of component B1 of RTV630 and 1.6 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1218 m/s, an acoustic impedance of 1.42 Mrayl, an acoustic attenuation of 8.70 dB/cm at 2 MHz, and a Shore A hardness of 58 HA.

### Example 4

(1) 80 g of polycaprolactone diol (Mn=2000), 5 g of hydroxyl-terminated polysiloxane (Mn=2000), 0.45 g of bismuth octoate, 1 g of BYK-A500, and 1,6-hexanediol, as a chain extender with a chain extension coefficient of 0.4, were mixed and stirred evenly and then dehydrated at 110° C. and a vacuum degree of −0.095 MPa for 2 hours to obtain a polyurethane prepolymer component A, in which the water content was less than 0.05 wt %.
(2) 55 g of dehydrated (i.e., free water removed) polycaprolactone diol (Mn=1000) was reacted with 40 g of methylene diphenyl diisocyanate (MDI) at 90° C. for 1.5 hours to obtain a polyurethane prepolymer component B with NCO %=9.5%.

After that, the polyurethane prepolymer components A and B were mixed in a ratio satisfying the isocyanate index R=0.9 to 1.1. In this example, I=0.95. Specifically, 10 g of the polyurethane prepolymer component A and 3.3 g of the polyurethane prepolymer component B were mixed at high speed for 2 minutes, then vacuum defoamed, and cured at 25° C. for 24 hours to obtain a polyurethane elastomer with a sound velocity of 1517 m/s, an acoustic impedance of 1.58 Mrayl, an acoustic attenuation of 5.79 dB/cm at 2 MHZ, and a Shore A hardness of 45 HA.

In the polyurethane elastomer, the molar ratio of the polysiloxane structural unit to polyurethane main chain structural unit was ranged from about 0.005:1 to about 0.05:1.
(3) 10 g of the component A1 of RTV630, 6 g of the polyurethane prepolymer component A prepared in step (1), and 0.75 g of silane coupling agent KH550 were mixed and stirred at high speed for 5 minutes, then 1 g of component B1 of RTV630 and 1.98 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1229 m/s, an acoustic impedance of 1.41 Mrayl, an acoustic attenuation of 8.25 dB/cm at 2 MHz, and a Shore A hardness of 55 HA.

### Example 5

(1) 65 g of polytetrahydrofuran diol (Mn=3000), 6.5 g of hydroxyl-terminated polysiloxane (Mn=2000), 0.45 g of bismuth octoate, 1 g of BYK-A500, and 1,6-hexanediol, as a chain extender with a chain extension coefficient of 0.3, were mixed and stirred evenly and then dehydrated at 110° C. and a vacuum degree of −0.095 MPa for 2 hours to obtain a polyurethane prepolymer component A, in which the water content was less than 0.05 wt %.
(2) 55 g of dehydrated (i.e., free water removed) polycaprolactone diol (Mn=2000) was reacted with 40 g of methylene diphenyl diisocyanate (MDI) at 90° C. for 2 hours to obtain a polyurethane prepolymer component B with NCO %=14.7%.

After that, the polyurethane prepolymer components A and B were mixed in a ratio satisfying the isocyanate index R=0.9 to 1.1. In this example, I=0.95. Specifically, 10 g of the polyurethane prepolymer component A and 4.5 g of the polyurethane prepolymer component B were mixed at high speed for 2 minutes, then vacuum defoamed, and cured at 25° C. for 24 hours to obtain a polyurethane elastomer with a sound velocity of 1535 m/s, an acoustic impedance of 1.61 Mrayl, an acoustic attenuation of 4.98 dB/cm at 2 MHz, and a Shore A hardness of 40 HA.

In the polyurethane elastomer, the molar ratio of the polysiloxane structural unit to polyurethane main chain structural unit was ranged from about 0.005:1 to about 0.06:1.
(3) 10 g of the component A1 of RTV630, 5 g of the polyurethane prepolymer component A prepared in step (1), and 0.75 g of silane coupling agent KH550 were mixed and stirred at high speed for 5 minutes, then 1 g of component B1 of RTV630 and 2.25 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1224 m/s, an acoustic impedance of 1.41 Mrayl, an acoustic attenuation of 8.03 dB/cm at 2 MHZ, and a Shore A hardness of 45 HA.

### Example 6

(1) 80 g of hydroxyl-terminated polybutadiene (Mn=1000), 8 g of hydroxyl-terminated polysiloxane (Mn-2000), 0.32 g of dibutyltin dilaurate, 1 g of BYK-A500, and 1,6-hexanediol, as a chain extender with a chain extension coefficient of 0.3, were mixed and stirred evenly and then dehydrated at 110° C. and a vacuum degree of −0.095 MPa for 2 hours to obtain a polyurethane prepolymer component A, in which the water content was less than 0.05 wt %.
(2) 65 g of dehydrated (i.e., free water removed) polyethylene glycol (Mn=2000) was reacted with 40 g of dicyclohexylmethane diisocyanate (HMDI) at 90° C. for 1 hour to obtain a polyurethane prepolymer component B with NCO %=15.85%.

After that, the prepolymer components A and B were mixed in a ratio satisfying the isocyanate index R=0.9 to 1.1. In this example, I=0.95. Specifically, 10 g of the polyurethane prepolymer component A and 10 g of the polyurethane prepolymer component B were mixed at high speed for 2 minutes, then vacuum defoamed, and cured at 40° C. for 24 hours to obtain a polyurethane elastomer with a sound velocity of 1588 m/s, an acoustic impedance of 1.60 Mrayl, an acoustic attenuation of 6.98 dB/cm at 2 MHz, and a Shore A hardness of 65 HA.

In the polyurethane elastomer, the molar ratio of the polysiloxane structural unit to polyurethane main chain structural unit was ranged from about 0.025:1 to about 0.15:1.
(3) 10 g of the component A1 of RTV630, 10 g of the polyurethane prepolymer component A prepared in step (1), and 1.5 g of silane coupling agent KH560 were mixed and stirred at high speed for 10 minutes, then 1 g of component B1 of RTV630 and 8 g of the polyurethane prepolymer component B prepared in step (2) were added. The mixture was evenly stirred at a high speed of 350-500 rpm, and then degassed. The degassed mixture was placed in a 40° C. air-blowing drying oven and cured for 24 hours to obtain polyurethane-modified silicone rubber with a sound velocity of 1386 m/s, an acoustic impedance of 1.49 Mrayl, an acoustic attenuation of 7.97 dB/cm at 2 MHZ, and a Shore A hardness of 62 HA.

### Example 7

(1) 75 g of polytetrahydrofuran diol (Mn=1000), 0.35 g of dibutyltin dilaurate, 1 g of BYK-A501, and 1,4-butanediol (BDO), as a chain extender with a chain extension coefficient of 0.5, were mixed and stirred evenly and then dehydrated at 110° C. and a vacuum degree of −0.095 MPa for 2 hours to obtain a polyurethane prepolymer component A, in which the water content was less than 0.05 wt %.
(2) 55 g of dehydrated (i.e., free water removed) polypropylene glycol (Mn=1500) was reacted with 38 g of dicyclohexylmethane diisocyanate (HMDI) at 80° C. for 2.5 hours to obtain a polyurethane prepolymer component B with NCO %=14.7%.

After that, the polyurethane prepolymer components A and B were mixed in a ratio satisfying the isocyanate index R=0.9 to 1.1. In this example, I=0.95. Specifically, 10 g of the polyurethane prepolymer component A and 6 g of the polyurethane prepolymer component B were mixed at high speed for 2 minutes, then vacuum defoamed, and cured at 25° C. for 24 hours to obtain a polyurethane elastomer with a sound velocity of 1541 m/s, an acoustic impedance of 1.59 Mrayl, an acoustic attenuation of 6.67 dB/cm at 2 MHz, and a Shore A hardness of 60 HA.
(3) 10 g of the component A1 of RTV630, 3.5 g of the polyurethane prepolymer component A prepared in step (1), and 0.6 g of silane coupling agent KH560 were mixed and stirred at high speed for 5 minutes, then 1 g of component B1 of RTV630 and 2.1 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1202 m/s, an acoustic impedance of 1.46 Mrayl, an acoustic attenuation of 12.95 dB/cm at 2 MHz, and a Shore A hardness of 60 HA.

TABLE 1

| | | Example | Example | Example | Example | Example | Example | Example |
|---|---|---|---|---|---|---|---|---|
| Product | | 1 | 2 | 3 | 4 | 5 | 6 | 7 |
| Polyurethane | Sound velocity/m/s | 1541 | 1573 | 1608 | 1517 | 1535 | 1588 | 1541 |
| elastomer | Acoustic impedance/ | 1.59 | 1.60 | 1.65 | 1.58 | 1.61 | 1.60 | 1.59 |
| | Mrayl | | | | | | | |
| | Acoustic attenuation | 6.55 | 7.57 | 6.08 | 5.79 | 4.98 | 6.98 | 6.67 |
| | at 2 MHz/dB/cm | | | | | | | |
| | Hardness (Shore A) | 60 | 60 | 55 | 45 | 40 | 65 | 60 |
| Polyurethane-modified | Sound velocity/m/s | 1190 | 1254 | 1218 | 1229 | 1224 | 1386 | 1202 |
| silicone rubber | Acoustic impedance/ | 1.45 | 1.43 | 1.42 | 1.41 | 1.41 | 1.49 | 1.46 |
| | Mrayl | | | | | | | |
| | Acoustic attenuation | 8.07 | 8.95 | 8.70 | 8.25 | 8.03 | 7.97 | 12.95 |
| | at 2 MHz/dB/cm | | | | | | | |
| | Hardness (Shore A) | 60 | 60 | 58 | 55 | 45 | 62 | 60 |

### Comparative Example 1

(1) 70 g of polytetrahydrofuran diol (Mn=4000), 7 g of hydroxyl-terminated polysiloxane (Mn=2000), 0.32 g of dibutyltin dilaurate, 1 g of BYK-A500, and 1,4-butanediol, as a chain extender with a chain extension coefficient of 0.25, were mixed and stirred evenly and then dehydrated at 110° C. and a vacuum degree of −0.095 MPa for 2 hours to obtain a polyurethane prepolymer component A, in which the water content was less than 0.05 wt %.
(2) 50 g of dehydrated (i.e., free water removed) polytetrahydrofuran diol (Mn=4000) was reacted with 35 g of dicyclohexylmethane diisocyanate (HMDI) at 90° C. for 1.5 hours to obtain a polyurethane prepolymer component B with NCO %=14.7%.

After that, the polyurethane prepolymer components A and B were mixed in a ratio satisfying the isocyanate index R=0.9 to 1.1. In this example, I=0.95. Specifically, 10 g of the polyurethane prepolymer component A and 4 g of the polyurethane prepolymer component B were mixed at high speed for 2 minutes, then vacuum defoamed, and cured at 50° C. for 24 hours to obtain a polyurethane elastomer with a sound velocity of 1516 m/s, an acoustic impedance of 1.58 Mrayl, and an acoustic attenuation of 4.22 dB/cm at 2 MHz. The Shore A hardness of the polyurethane elastomer was 20 HA, at which the polyurethane elastomer is a gel and cannot be used as an acoustic lens material.

In the polyurethane elastomer, the molar ratio of the polysiloxane structural unit to polyurethane main chain structural unit was ranged from about 0.005:1 to about 0.04:1.
(3) 10 g of the component A1 of RTV630, 8 g of the polyurethane prepolymer component A prepared in step (1), and 1.5 g of silane coupling agent KH560 were mixed and stirred at high speed for 10 minutes, then 1 g of component B1 of RTV630 and 3.2 g of the polyurethane prepolymer component B prepared in step (2) were added. The mixture was evenly stirred at a high speed of 350-500 rpm, and then degassed. The degassed mixture was placed in a 40° C. air-blowing drying oven and cured for 24 hours to obtain polyurethane-modified silicone rubber with a sound velocity of 1298 m/s, an acoustic impedance of 1.49 Mrayl, and an acoustic attenuation of 7.10 dB/cm at 2 MHz. The Shore A hardness of the polyurethane elastomer was merely 35 HA. The modified silicone rubber prepared in this comparison example has poor hardness, which may not meet the mechanical requirements such as wear resistance needed for acoustic lens materials, thus cannot be used in preparation of an acoustic lens.

### Comparative Example 2

(1) 70 g of a poly(ethylene adipate) diol (Mn=1000), whose molecular formula is

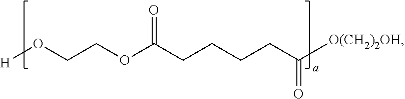

where a is a positive integer, 7 g of hydroxyl-terminated polysiloxane (Mn=2000), 0.35 g of dibutyltin dilaurate, 1 g of BYK-A501, and 1,4-butanediol (BDO), as a chain extender with a chain extension coefficient of 0.5, were mixed and stirred evenly and then dehydrated at 110° C. and a vacuum degree of −0.095 MPa for 2 hours to obtain a polyurethane prepolymer component A, in which the water content was less than 0.05 wt %.
(2) 55 g of a dehydrated (i.e., free water removed) trimethylolpropane polyether polyol (Mn=2000), whose monomer formula is

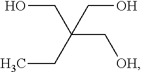

was reacted with 45 g of dicyclohexylmethane diisocyanate (HMDI) at 80° C. for 3 hours to obtain a polyurethane prepolymer component B with NCO %=14.7%.

After that, the polyurethane prepolymer components A and B were mixed in a ratio satisfying the isocyanate index R=0.9 to 1.1. In this example, I=0.95. Specifically, 10 g of the polyurethane prepolymer component A and 6 g of the polyurethane prepolymer component B were mixed at high speed for 2 minutes, then vacuum defoamed, and cured at 25° C. for 24 hours to obtain a polyurethane elastomer with a sound velocity of 1671 m/s, an acoustic impedance of 1.65 Mrayl, an acoustic attenuation of 16.75 dB/cm at 2 MHz, and a Shore A hardness of 65 HA.

In the polyurethane elastomer, the molar ratio of the polysiloxane structural unit to polyurethane main chain structural unit was ranged from about 0.025:1 to about 0.15:1.
(3) 10 g of the component A1 of RTV630, 3.5 g of the polyurethane prepolymer component A prepared in step (1), and 0.6 g of silane coupling agent KH560 were mixed and stirred at high speed for 5 minutes, then 1 g of component B1 of RTV630 and 2.1 g of the polyurethane prepolymer component B prepared in step (2) were added. The mixture was evenly stirred at a high speed of 350-500 rpm, and then degassed. The degassed mixture was placed in a 25° C. air-blowing drying oven and cured for 24 hours to obtain polyurethane-modified silicone rubber with a sound velocity of 1359 m/s, an acoustic impedance of 1.50 Mrayl, an acoustic attenuation of 24.51 dB/cm at 2 MHZ, and a Shore A hardness of 60 HA.

TABLE 2

| | | Comparative | Comparative |
|---|---|---|---|
| Product | | Example 1 | Example 2 |
| Polyurethane elastomer | Sound velocity/m/s | 1516 | 1671 |
| | Acoustic impedance/Mrayl | 1.58 | 1.65 |
| | Acoustic attenuation at 2 MHz/ | 4.22 | 16.75 |
| | dB/cm | | |
| | Hardness (Shore A) | 20 | 65 |
| Polyurethane-modified | Sound velocity/m/s | 1298 | 1359 |
| silicone rubber | Acoustic impedance/Mrayl | 1.49 | 1.50 |
| | Acoustic attenuation at 2 MHz/ | 7.10 | 24.51 |
| | dB/cm | | |
| | Hardness (Shore A) | 35 | 60 |

From Tables 1 and 2, it can be seen that:
(1) The polyurethane elastomer in the present application exhibits excellent acoustic performance, with a sound velocity of 1500 m/s to 1650 m/s, acoustic impedance of 1.50 Mrayl to 1.70 Mrayl, and acoustic attenuation of 4.00 dB/cm to 8.00 dB/cm at 2 MHz.
(2) The polyurethane-modified silicone rubber in the present application also exhibits excellent acoustic performance, with a sound velocity of 1000 m/s to 1500 m/s, acoustic impedance of 1.40 Mrayl to 1.50 Mrayl, and acoustic attenuation of 5.00 dB/cm to 15.00 dB/cm at 2 MHz. Particularly, when the polyurethane contains polysiloxane structural units, the acoustic attenuation of the polyurethane-modified silicone rubber can be 5.00 dB/cm to 8.00 dB/cm.
(3) As shown in Comparative Example 1, when the chain extension coefficient of the chain extender is less than 0.3, the polyurethane elastomer is in gel form and cannot be used as an acoustic lens material. The polyurethane-modified silicone rubber exhibits poor hardness, which may not meet the mechanical requirements such as wear resistance needed for acoustic lens materials, thus cannot be used in preparation of an acoustic lens.
(4) As shown in Comparative Example 2, when the polyol segments in the polyurethane elastomer are changed, the acoustic attenuation of the polyurethane elastomer increases to 16.75 dB/cm. With respect to a sound with a frequency of 2 MHZ, the acoustic attenuation of the polyurethane-modified silicone rubber increases to 24.51 dB/cm.

The technical features of the above-mentioned embodiments can be combined arbitrarily. In order to make the description concise, not all possible combinations of the technical features are described in the embodiments. However, as long as there is no contradiction in the combination of these technical features, the combinations should be considered as in the scope of the present disclosure.

The above-described embodiments are only several implementations of the present disclosure, and the descriptions are relatively specific and detailed, but they should not be construed as limiting the scope of the present disclosure. It should be understood by those of ordinary skill in the art that various modifications and improvements can be made without departing from the concept of the present disclosure, and all fall within the protection scope of the present disclosure. Therefore, the patent protection of the present disclosure shall be defined by the appended claims.

What is claimed is:

1. A polyurethane elastomer comprising a structural unit represented by formula I and a chain extender unit, wherein the structural unit represented by formula I is a polyurethane main chain structural unit: 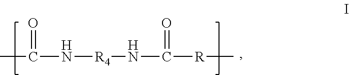
where R₄ is C6-C20 arylene or C6-C20 cycloalkylene, and R is selected from structural units represented by formula I-1 and/or formula I-2 and/or formula I-3: 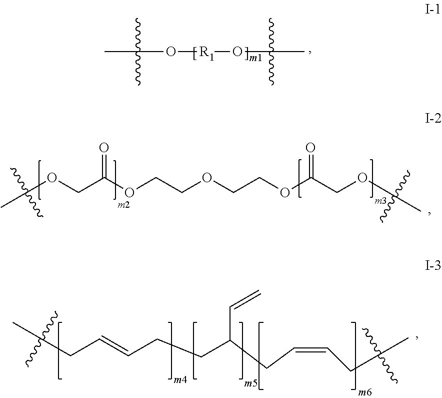
where R₁, R₂, and R₃ are each independently selected from C2-C10 hydrocarbon chains, and m1, m2, m3, m4, m5, and m6 are all positive integers;
a number average molecular weight of the structural unit represented by formula I-1 is ranged from 400 to 5000, a number average molecular weight of the structural unit represented by formula I-2 is ranged from 400 to 5000, a number average molecular weight of the structural unit represented by formula I-3 is ranged from 400 to 5000; and
a molar ratio of the chain extender unit to the structural unit represented by formula I is ranged from 0.3:1 to 0.8:1.

2. The polyurethane elastomer according to claim 1, further comprising a structural unit represented by formula II: 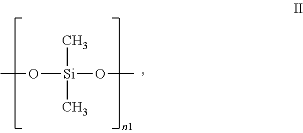
wherein the structural unit represented by formula II is a polysiloxane structural unit, a number average molecular weight of the polysiloxane structural unit is ranged from 500 to 6000, and n1 is a positive integers; and
a molar ratio of the structural unit represented by formula II to the structural unit represented by formula I is ranged from 0.001:1 to 0.2:1.

3. The polyurethane elastomer according to claim 1, wherein the hydrocarbon chains of R₁, R₂, and/or R₃ comprises at least one unsaturated bond; and/or
the number of side chains in at least one of the hydrocarbon chains of R₁, R₂, and R₃ is smaller than or equal to 2; and/or
the number of carbon atoms in each side chain of the hydrocarbon chains of R₁, R₂, and R₃ is smaller than or equal to 2.

4. The polyurethane elastomer according to claim 1, wherein R₁ is selected from the group consisting of —CH₂CH₂CH₂CH₂—, —CH₂CH₂—, —CH(CH₃)CH₂—, —CH₂CH—CHCH₂—; and/or
R₂ is —CH₂CH₂CH₂CH₂CH₂—; and/or
R₃ is —CH₂CH₂CH₂CH₂CH₂—; and/or
R₄ is selected from the group consisting of:

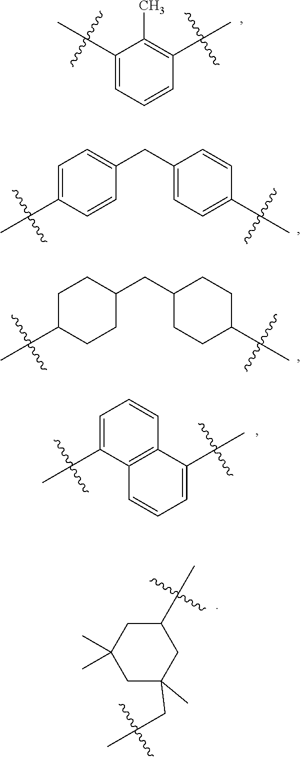

5. The polyurethane elastomer according to claim 2, wherein a molar ratio of the structural unit represented by formula II to the structural unit represented by formula I is ranged from 0.005:1 to 0.2:1.

6. The polyurethane elastomer according to claim 2, wherein the number average molecular weight of the polysiloxane structural unit is ranged from 1000 to 3000.

7. The polyurethane elastomer according to claim 1, wherein the chain extender unit is a structural unit represented by 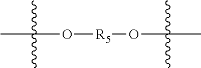
(formula III), where R₅ is selected from C3-C10 hydrocarbon chains;
a molar ratio of the chain extender unit to the structural unit represented by formula I is ranged from 0.3:1 to 0.6:1.

8. A raw material mixture for preparing a polyurethane elastomer, comprising a first prepolymer component and a second prepolymer component;
the first prepolymer component comprises a first raw material A, a catalyst, and a chain extender with a chain extension coefficient ranged from 0.3 to 0.8;
the first prepolymer component and the second prepolymer component satisfy an isocyanate index R ranged from 0.8 to 1.2;
where:
the first raw material A comprises a raw material of formula IV-1, formula IV-2 or formula IV-3: 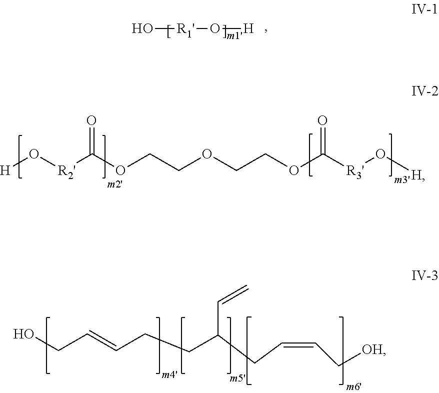
where R₁′, R₂′, and R₅′ are each independently selected from C2-C10 hydrocarbon chains, m1′, m2′, and m3′ are all positive integers, and m4′, m5′, and m6′ are all positive integers;
a number average molecular weight of the raw material of formula IV-1, formula IV-2 or formula IV-3 is ranged from 400 to 5000;
the structure of the second prepolymer component is represented by formula V: 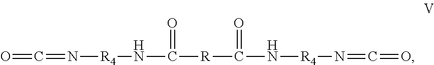
where R₄ is C6-C20 arylene or C6-C20 cycloalkylene, and R is selected from structural units represented by formula I-1 and/or formula I-2 and/or formula I-3: 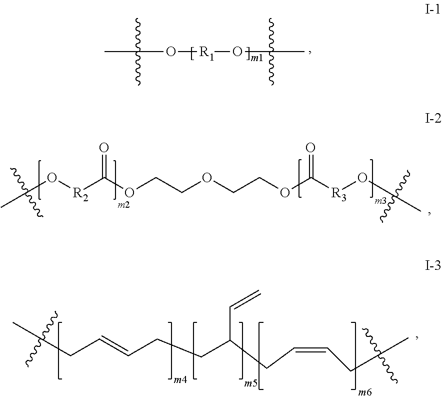
where R₁, R₂, and R₃ are each independently selected from C2-C10 hydrocarbon chains, and m1, m2, m3, m4, m5, and m6 are all positive integers; and
a number average molecular weight of the structural unit represented by formula I-1 is ranged from 400 to 5000, and a number average molecular weight of the structural unit represented by formula I-2 is ranged from 400 to 5000, and a number average molecular weight of the structural unit represented by formula I-3 is ranged from 400 to 5000.

9. The raw material mixture according to claim 8, wherein the first prepolymer component further comprises a hydroxyl-terminated polysiloxane, a number average molecular weight of the hydroxyl-terminated polysiloxane is ranged from 500 to 6000.

10. The raw material mixture according to claim 8, wherein the number average molecular weight of the raw material of formula IV-1 or formula IV-2 is ranged from 500 to 4000.

11. The raw material mixture according to claim 8, wherein the chain extension coefficient of the chain extender is ranged from 0.3 to 0.6.

12. The raw material mixture according to claim 8, wherein the chain extender is selected from the group consisting of 1,4-butanediol, 1,6-hexanediol, glycerol, trimethylolpropane, sorbitol, and combinations thereof.

13. The raw material mixture according to claim 8, wherein the isocyanate index R is ranged from 0.9 to 1.1.

14. The raw material mixture according to claim 8, wherein the raw material of formula IV-1, formula IV-2 or formula IV-3 is selected from the group consisting of:
polytetrahydrofuran diol represented by 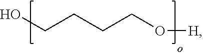
polycaprolactone diol represented by 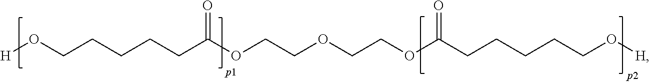
polyethylene glycol represented by 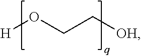
polypropylene glycol represented by 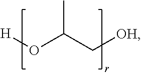
hydroxyl-terminated polybutadiene, and combinations thereof;
where o, p1, p2, q, and r are each a positive integer and greater than or equal to 1.

15. The raw material mixture according to claim 8, wherein the second prepolymer component is polymerized from 40 to 70 parts by mass of a second raw material B and 25 to 50 parts by mass of an diisocyanate;
the second raw material B comprises a raw material of formula IV-1 or formula IV-2 or formula IV-3: 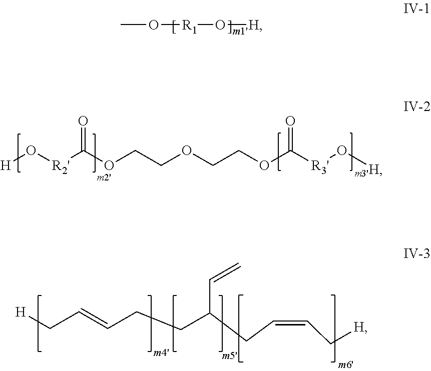
where R₁′, R₂′, and R₃′ are each independently selected from C2-C10 hydrocarbon chains, and m1′, m2′, m3′, m4′, m5′, and m6′ are all positive integers.

16. A polyurethane-modified silicone rubber comprising:
a silicone rubber base material; and
a polyurethane elastomer according to claim 1,
wherein the polyurethane elastomer is dispersed in the silicone rubber base material.

17. The polyurethane-modified silicone rubber according to claim 16, being prepared by:
mixing raw materials for silicone rubber, a raw material mixture for the polyurethane elastomer, and a compatibilizer to form a mixture; and
curing the mixture to obtain a shaped product;
wherein the raw materials for silicone rubber comprise a first silicone rubber component C1 and a second silicone rubber component C2, wherein:
a weight ratio of the first silicone rubber component C1 to the raw material mixture for the polyurethane elastomer is ranged from 10:1.25 to 10:25;
a weight ratio of the first silicone rubber component C1 to the compatibilizer is ranged from 10:0.125 to 10:7.5;
a weight ratio of the first silicone rubber component C1 to the second silicone rubber component C2 is ranged from 10:0.5 to 10:5;
the first silicone rubber component C1 comprises vinyl silicone rubber and a crosslinking agent, wherein the structure of the vinyl silicone rubber is represented by formula VI, and R₁ₐ, R_{1b}, R_{1c}, R_{1d} are each independently selected from H, substituted or unsubstituted C1-C5 linear or branched alkyl groups, substituted or unsubstituted C6-C20 aromatic groups, n2≥1000; 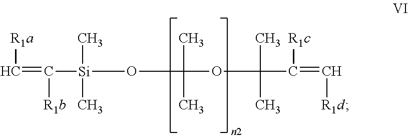
the crosslinking agent contains a silicon-hydrogen bond;
the second silicone rubber component C2 comprises a catalyst capable of catalyzing an addition reaction between the vinyl silicone rubber represented by formula VI and the crosslinking agent.

18. The polyurethane-modified silicone rubber according to claim 17, wherein the weight ratio of the first silicone rubber component C1 to the raw material mixture for the polyurethane elastomer is ranged from 10:2.0 to 10:20.0; and/or
a weight ratio of the first silicone rubber component C1 to the first prepolymer component in the raw material mixture for the polyurethane elastomer is ranged from 10:3.5 to 10:10; and/or
the weight ratio of the first silicone rubber component C1 to the compatibilizer is ranged from 10:0.5 to 10:5; and/or
a weight ratio of the first silicone rubber component C1 to the second prepolymer component in the raw material mixture for the polyurethane elastomer is ranged from 10:0.5 to 10:10.

19. An acoustic lens comprising the polyurethane elastomer according to claim 1.

20. An ultrasonic probe comprising the acoustic lens according to claim 19.
